SEQUENCE LISTING (66 sequences)

SEQ ID NO: 1 - protein, 5 aa, Artificial Sequence
  Synthetic sequence R12 antibody CDRH1
AYYMS

SEQ ID NO: 2 - protein, 17 aa, Artificial Sequence
  Synthetic sequence R12 antibody CDRH2
TIYPSSGKTYYATWVNG

SEQ ID NO: 3 - protein, 12 aa, Artificial Sequence
  Synthetic sequence R12 antibody CDRH3
DSYADDGALFNI

SEQ ID NO: 4 - protein, 12 aa, Artificial Sequence
  Synthetic sequence R12 antibody CDRL1
TLSSAHKTDTID

SEQ ID NO: 5 - protein, 7 aa, Artificial Sequence
  Synthetic sequence R12 antibody CDRL2
GSYTKRP

SEQ ID NO: 6 - protein, 9 aa, Artificial Sequence
  Synthetic sequence R12 antibody CDRL3
GADYIGGYV

SEQ ID NO: 7 - protein, 121 aa, Artificial Sequence
  Synthetic sequence R12 antibody VH
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 8 - protein, 112 aa, Artificial Sequence
  Synthetic sequence R12 antibody VL
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 9 - protein, 248 aa, Artificial Sequence
  Synthetic sequence R12 scFv
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 10 - protein, 5 aa, Artificial Sequence
  Synthetic sequence 2A2 antibody CDRH1
DYEMH

SEQ ID NO: 11 - protein, 17 aa, Artificial Sequence
  Synthetic sequence 2A2 antibody CDRH2
AIDPETGGTAYNQKFKG

SEQ ID NO: 12 - protein, 9 aa, Artificial Sequence
  Synthetic sequence 2A2 antibody CDRH3
YYDYDSFTY

SEQ ID NO: 13 - protein, 11 aa, Artificial Sequence
  Synthetic sequence 2A2 antibody CDRL1
KASQNVDAAVA

SEQ ID NO: 14 - protein, 7 aa, Artificial Sequence
  Synthetic sequence 2A2 antibody CDRL2
SASNRYT

SEQ ID NO: 15 - protein, 9 aa, Artificial Sequence
  Synthetic sequence 2A2 antibody CDRL3
QQYDIYPYT

SEQ ID NO: 16 - protein, 118 aa, Artificial Sequence
  Synthetic sequence 2A2 antibody VH
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 17 - protein, 107 aa, Artificial Sequence
  Synthetic sequence 2A2 antibody VL
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 18 - protein, 237 aa, Artificial Sequence
  Synthetic sequence 2A2 scFv
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAGGGSGGGSGGGSDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 19 - protein, 5 aa, Artificial Sequence
  Synthetic sequence R11 antibody CDRH1
DYPIS

SEQ ID NO: 20 - protein, 16 aa, Artificial Sequence
  Synthetic sequence R11 antibody CDRH2
FINSGGSTWYASWVKG

SEQ ID NO: 21 - protein, 11 aa, Artificial Sequence
  Synthetic sequence R11 antibody CDRH3
GYSTYYGDFNI

SEQ ID NO: 22 - protein, 11 aa, Artificial Sequence
  Synthetic sequence R11 antibody CDRL1
QASQSIDSNLA

SEQ ID NO: 23 - protein, 7 aa, Artificial Sequence
  Synthetic sequence R11 antibody CDRL2
RASNLAS

SEQ ID NO: 24 - protein, 12 aa, Artificial Sequence
  Synthetic sequence R11 antibody CDRL3
LGGVGNVSYRTS

SEQ ID NO: 25 - protein, 116 aa, Artificial Sequence
  Synthetic sequence R11 antibody VH
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISS

SEQ ID NO: 26 - protein, 110 aa, Artificial Sequence
  Synthetic sequence R11 antibody VL
ELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 27 - protein, 241 aa, Artificial Sequence
  Synthetic sequence R11 scFv
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSGGGGSGGGGSGGGGSELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 28 - protein, 5 aa, Artificial Sequence
  Synthetic sequence Y31 antibody CDRL1
SHWMS

SEQ ID NO: 29 - protein, 16 aa, Artificial Sequence
  Synthetic sequence Y31 antibody CDRH2
IIAASGSTYYANWAKG

SEQ ID NO: 30 - protein, 12 aa, Artificial Sequence
  Synthetic sequence Y31 antibody CDRH3
DYGDYRLVTFNI

SEQ ID NO: 31 - protein, 11 aa, Artificial Sequence
  Synthetic sequence Y31 antibody CDRL1
QASQSIGSYLA

SEQ ID NO: 32 - protein, 7 aa, Artificial Sequence
  Synthetic sequence Y31 antibody CDRL2
YASNLAS

SEQ ID NO: 33 - protein, 9 aa, Artificial Sequence
  Synthetic sequence Y31 antibody CDRL3
LGSLNSDNV

SEQ ID NO: 34 - protein, 6 aa, Artificial Sequence
  Synthetic sequence anti-ROR1 scFv CDRH1
DTIDWY

SEQ ID NO: 35 - protein, 16 aa, Artificial Sequence
  Synthetic sequence anti-ROR1 scFv CDRH2
VQSDGSYTKRPGVPDR

SEQ ID NO: 36 - protein, 8 aa, Artificial Sequence
  Synthetic sequence anti-ROR1 scFv CDRH3
YIGGYVFG

SEQ ID NO: 37 - protein, 11 aa, Artificial Sequence
  Synthetic sequence anti-ROR1 scFv CDRL1
ASGFDFSAYYM

SEQ ID NO: 38 - protein, 7 aa, Artificial Sequence
  Synthetic sequence anti-ROR1 scFv CDRL2
TIYPSSG

SEQ ID NO: 39 - protein, 9 aa, Artificial Sequence
  Synthetic sequence anti-ROR1 scFv CDRL3
ADRATYFCA

SEQ ID NO: 40 - protein, 5 aa, Artificial Sequence
  Synthetic sequence anti-ROR1 scFv CDRH1
SNLAW

SEQ ID NO: 41 - protein, 16 aa, Artificial Sequence
  Synthetic sequence anti-ROR1 scFv CDRH2
RASNLASGVPSRFSGS

SEQ ID NO: 42 - protein, 8 aa, Artificial Sequence
  Synthetic sequence anti-ROR1 scFv CDRH3
NVSYRTSF

SEQ ID NO: 43 - protein, 11 aa, Artificial Sequence
  Synthetic sequence anti-ROR1 scFv CDRL1
SGSDINDYPIS

SEQ ID NO: 44 - protein, 7 aa, Artificial Sequence
  Synthetic sequence anti-ROR1 scFv CDRL2
INSGGST

SEQ ID NO: 45 - protein, 8 aa, Artificial Sequence
  Synthetic sequence anti-ROR1 scFv CDRL3
YFCARGYS

SEQ ID NO: 46 - protein, 12 aa, Artificial Sequence
  Synthetic sequence amino acid spacer
ESKYGPPCPPCP

SEQ ID NO: 47 - protein, 12 aa, Artificial Sequence
  Synthetic sequence amino acid spacer
ESKYGPPCPSCP

SEQ ID NO: 48 - protein, 15 aa, Artificial Sequence
  Synthetic sequence amino acid spacer
GGGGSGGGGSGGGGS

SEQ ID NO: 49 - protein, 327 aa, Artificial Sequence
  Synthetic sequence IgG4 Fc
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 18 aa, Artificial Sequence
  Synthetic sequence Thosea asigna virus 2A (T2A) self-cleaving element
EGRGSLLTCGDVEENPGP

SEQ ID NO: 51 - protein, 21 aa, Artificial Sequence
  Synthetic sequence T2A, modified
GSGEGRGSLLTCGDVEENPGP

SEQ ID NO: 52 - protein, 19 aa, Artificial Sequence
  Synthetic sequence Porcine teschovirus-1 2A (P2A) self-cleaving element
ATNFSLLKQAGDVEENPGP

SEQ ID NO: 53 - protein, 22 aa, Artificial Sequence
  Synthetic sequence P2A, modified
GSGATNFSLLKQAGDVEENPGP

SEQ ID NO: 54 - protein, 21 aa, Artificial Sequence
  Synthetic sequence Equine rhinitis A virus 2A (E2A) self-cleaving element
QCTNYALLKLAGSDVESNPGP

SEQ ID NO: 55 - protein, 24 aa, Artificial Sequence
  Synthetic sequence E2A, modified
GSGQCTNYALLKLAGSDVESNPGP

SEQ ID NO: 56 - protein, 22 aa, Artificial Sequence
  Synthetic sequence foot-and-mouth disease virus 2A (F2A) self-cleaving element
VKQTLNFDLLKLAGDVESNPGP

SEQ ID NO: 57 - protein, 25 aa, Artificial Sequence
  Synthetic sequence T2A, modified
GSGVKQTLNFDLLKLAGDVESNPGP

SEQ ID NO: 58 - DNA, 25 bp, Artificial Sequence
  Synthetic sequence TRAC_sgRNA_pLenti_F1, nucleotide
caccggagaatcaaaatcggtgaat

SEQ ID NO: 59 - DNA, 25 bp, Artificial Sequence
  Synthetic sequence TRAC_sgRNA_pLenti_R1
aaacattcaccgattttgattctcc

SEQ ID NO: 60 - DNA, 24 bp, Artificial Sequence
  Synthetic sequence PD1_sgRNA_F1
caccgcagttgtgtgacacggaag

SEQ ID NO: 61 - DNA, 24 bp, Artificial Sequence
  Synthetic sequence PD1_sgRNA_R1
aaaccttccgtgtcacacaactgc

SEQ ID NO: 62 - DNA, 25 bp, Artificial Sequence
  Synthetic sequence CTLA4_sgRNA_F1
caccggcaaaggtgagtgagacttt

SEQ ID NO: 63 - DNA, 25 bp, Artificial Sequence
  Synthetic sequence CTLA4_sgRNA_R1
aaacaaagtctcactcacctttgcc

SEQ ID NO: 64 - DNA, 25 bp, Artificial Sequence
  Synthetic sequence LAG3_sgRNA_F1
caccggtttctgcagccgctttggg

SEQ ID NO: 65 - DNA, 25 bp, Artificial Sequence
  Synthetic sequence LAG3_sgRNA_R2
aaaccccaaagcggctgcagaaacc

SEQ ID NO: 66 - DNA, 72 bp, Artificial Sequence
  Synthetic sequence
ctcgagggcggcggagagggcagaggaagtcttctaacatgcggtgacgtggaggagaatcccggccctagg

(19) United States
(12) Patent Application Publication
(10) Pub. No.: US 2021/0069241 A1
(43) Pub. Date: Mar. 11, 2021

(54) COMPOSITIONS AND METHODS OF IMMUNOTHERAPY TARGETING TIGIT AND/OR CD112R OR COMPRISING CD226 OVEREXPRESSION

(71) Applicant: FRED HUTCHINSON CANCER RESEARCH CENTER, Seattle, WA (US)

(72) Inventors: Stanley R. RIDDELL, Sammamish, WA (US); Susanna Carolina BERGER, Seattle, WA (US); Shivani SRIVASTAVA, Seattle, WA (US)

(21) Appl. No.: 16/757,670

(22) PCT Filed: Oct. 19, 2018
(86) PCT No.: PCT/US2018/056796

§ 371 Date: Apr. 20, 2020

Related U.S. Application Data
(60) Provisional application No. 62/575,326, filed on Oct. 20, 2017.

(51) Int. Cl. A61K 35/17; A61P 35/00; C07K 14/705; C07K 14/725

(52) U.S. Cl. CPC A61K 35/17; C07K 14/7051; C07K 14/70596; A61P 35/00

(57) ABSTRACT

The present disclosure provides compositions for treating solid tumors, including modified immune cells that specifically target tumor-associated antigens for immunotherapies, wherein expression or activity of T cell immunoreceptor with Ig and ITIM domains (TIGIT) and/or CD112R in the modified immune cells is inhibited to improve antitumor functionality of the cells. Alternatively, CD226 is overexpressed in such modified immune cells. Also provided are methods for treating solid tumors, such as tumors formed by common epithelial cancers.

### STATEMENT OF GOVERNMENT INTEREST

This invention was made with government support under CA114536 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_456WO_SEQUENCE_LISTING.txt. The text file is 26.4 KB, was created on Oct. 18, 2018, and is being submitted electronically via EFS-Web.

### BACKGROUND

Adoptive transfer of genetically modified immune cells has emerged as a potent therapy for various malignancies. For example, current modalities of adoptive T cell therapy include cells modified to express receptors specific for cancer antigens, such as chimeric antigen receptors (CARs) and high-affinity T cell receptors (TCRs). See, e.g., Harris and Kranz, Trends Pharmacol. Sci. 37(3):220 (2016). Upon exposure to the cancer antigen, the modified T cells exhibit cytolytic activity and/or send signals to initiate an immune response against the cancer.

In adoptive T cell therapies, modified T cells are typically activated by exposure to the cognate antigen in vitro or ex vivo, expanded, and then administered to the subject, where they proliferate and have anticancer activity. Trials using CAR-modified T cells (CAR-T cells) specific for the CD19 molecule on B-cell malignancies demonstrated marked disease regression in a subset of patients with advanced cancers (Barrett et al., 2014; Sadelain et al., 2013; Kalos et al., Sci. Transl. Med. 3:95ra73, 2011; Kochenderfer and Rosenberg, Nat. Rev. Clin. Oncol. 10:267, 2013). However, extending this therapy to solid tumors poses several challenges. For example, overstimulation due to prolonged antigen recognition and exposure to inflammatory signals can cause the T cells to lose effector function, a phenomenon called “T cell exhaustion.” See, e.g., Ghoneim et al., Trends Mol. Med. 22(12):1001 (2016). Additionally, the tumor microenvironment elicits a number of tolerance and immunosuppression mechanisms that can reduce the effectiveness of adoptive cell therapies. See, e.g., Draghiciu et al., Clin. Dev. Immunol. 439053 (2011).

Accordingly, new strategies are needed in adoptive cell therapies for solid tumors. The presently disclosed embodiments address these needs and provide other related advantages.

### BRIEF DESCRIPTION OF THE DRAWINGS

FIGS. 1A-1F show the frequency of CD4⁺ and CD8⁺ ROR1-specific CAR T cells in blood from two patients (X475 (A-C) and X461 (D-F) with ROR1⁺ cancer at various times following adoptive transfer of the CAR T cells. (A, D) Flow cytometry data showing the percentage of the CAR T cells at indicated time points, as indicated by the presence of a truncated EGFR (EGFRt) transduction marker. (B, E) Absolute numbers of EGFRt-positive T cells per microliter of blood at the indicated time points. (C, F) Quantitative PCR data showing the number of ROR1 CAR transgene copies per 1×10⁶ cells in blood at the indicated time points.

FIGS. 2A-2F show the frequency of CD4⁺ (A-C) and CD8⁺ T cells (D-F) that express the indicated co-inhibitory molecules in: patient blood that was used to manufacture the (A, D) ROR1-specific CAR T cells; (B, E) ROR1 CAR T cell-containing cell product; and (C, F) patient PBMC containing CAR T cells and endogenous T cells on day 14 after CAR T cell infusion. Expression was measured by flow cytometry.

FIGS. 3A and 3B show the frequency of (A) CD4⁺ and (B) CD8⁺ T cells that express the indicated co-inhibitory molecules in the CAR T cell product and in CAR T cells in a patient at the peak of expansion after infusion (“post tx”; grey bars), as measured by flow cytometry. FIGS. 3C and 3D show gene expression profiling of (C) CD4⁺ and (D) CD8⁺ T cells, indicating the fold difference in transcript abundance of the indicated co-inhibitory mRNAs in blood after treatment and in the ROR-1 CAR T cell product, as indicated in the figure keys.

FIG. 4 shows the frequency of CD19-targeting CART cells (“+”) and endogenous non CAR-expressing T cells (“−”) in patient blood that express TIGIT after adoptive transfer to treat CD19⁺ B cell malignancies.

FIG. 5A shows intracellular cytokine staining (IFN-γ; x-axes of graphs) of PBMC from a patient treated with ROR1 CAR T cells (CD4⁺ T cells, left-hand panels; CD8⁺ T cells, right-hand panels) following stimulation in vitro; specifically, cytokine staining was performed following stimulation of PBMC with: K562 cells (“K/mock”); K562 cells expressing ROR1 (“K/ROR1”); or with PMA/ionomycin (“PMA/Iono”; positive control). FIGS. 5B-5E show production of cytokines (B) IL-2; (C) TNF-α; (D) IFN-γ; and (E) GM-CSF by T cells as measured in culture supernatants from ROR1 CAR T cells prior to infusion into patients (“T cell”; left-hand side of vertical dividing line in graph) and from patient PBMC at the peak frequency post-infusion (“PBMC”; right-hand side of vertical dividing line in graph). CAR T cells and post-infusion PBMC were cultured with media, K562 cells (“K/m”), K562 cells expressing ROR1 (“K/R”), and ROR1⁺ Raji cells.

FIG. 5F shows expression of ligands for TIGIT (CD112, CD155) and CD112R (CD112) on K562 and K/R cells, as measured by flow cytometry.

FIGS. 6A and 6B show expression of CD226 on CD4⁺ and CD8⁺ ROR1 CAR T cells from (A) patient X461 and (6) patient X475. Cells were gated for EGFRt.

FIGS. 7A and 7B show IFN-γ production by (A) CD4⁺ and (B) CD8⁺ ROR1 4-1BB/CD3ζ and ROR1 CD28/CD3ζ CAR T cells with endogenous TIGIT expression (“R12:4-1BBz”; “R12:CD28”) or that were engineered to constitutively overexpress TIGIT (“R12:4-1BBz:TIGIT”; “R12:CD28:TIGIT”). Cytokine production was evaluated following stimulation of CAR T cells in culture with: K562 cells (control); K/R cells; cells from the ROR1-expressing MDA-MB-231 breast cancer cell line; or PMA/ionomycin (P/I).

FIGS. 8A-8E show data from an in vivo xenograft experiment in which mice were genetically engineered to develop human ROR1⁺ (hROR1⁺) tumors. (A) Magnetic Resonance Imaging (MM) of hROR1⁺ mice treated with a combination of cyclophosphamide and either control T cells expressing a tCD19 transduction marker or T cells expressing ROR1 4-1BB/CD3ζ CAR. MM were taken at 10 weeks (pre-treatment) and 16 weeks (post-treatment) following induction of ROR1⁺ tumors. Tumors are outlined and, in MM from the mice treated with ROR1 CAR T cells, indicated with white arrows. (B) Tumor nodule volumes of the indicated treatment groups measured over the course of the study. Cyclophosphamide and T cells were administered at the indicated time points (y-axis). Expression of the co-inhibitory molecules PD-1 (solid line with circles) and TIGIT (dashed line with triangles) by indicated T cells following adoptive transfer, as measured by mean fluorescence intensity (MFI; (C, D)) and flow cytometry (E).

### DETAILED DESCRIPTION

In certain aspects, the present disclosure provides methods and compositions for treating solid tumors using adoptive cell therapy approaches in which expression or activity of TIGIT and/or CD112R is inhibited and a tumor antigen is specifically targeted. By way of background, and without wishing to be bound by theory, certain immune cell activities may be modulated by agonistic and/or antagonistic signals originating in the extracellular environment. For example, signaling by pro-inflammatory cytokines (e.g., IL-1, IL-12, IL-18, TNF, and IFN-γ) can upregulate certain immune response mechanisms, while anti-inflammatory cytokines (e.g., IL-4, IL-6, and IL-10) can provide down-regulatory effects. Translation of such agonistic or antagonistic signals into a cellular immune response (or lack of a response) can be modulated by receptors that are expressed on the surface of immune cells.

TIGIT (T cell immunoreceptor with Ig and ITIM domains, also known as WUCAM and Vstm3) is an immunoreceptor expressed by immune cells including T cells, Natural Killer (NK) cells, and NK-T cells. See, e.g, Zhang et al., Cancer Immunol. Immunother. 65(3):305 (2015). TIGIT comprises an extracellular binding domain and an intracellular ITIM (immunoreceptor tyrosine-based inhibitory motif), and plays a role in cell-cell adhesion by binding to CD155 (also called PVR) and CD112 (also called PVRL2), which are expressed by certain cells to which a TIGIT-epxressing cell binds. A receptor that can partner with TIGIT, termed CD112R (also called PVRIG), is also expressed by T cells and binds to CD112. Binding of TIGIT to either of CD112 or CD115, or binding of CD112R to CD112, can in some aspects initiate an immunosuppressive signal that can reduce proliferation, decrease secretion of pro-inflammatory cytokines (e.g., IL-12B) and increase secretion of anti-inflammatory cytokines (e.g., IL-10) by the TIGIT-expressing cell. See, e.g., Lozano et al., J. Immunol. 188(8):3869 (2012); Zhu et al., J. Exp. Med. 213(2):167-176, 2016.

In some aspects, embodiments provided in the present disclosure, are based upon the unexpected finding that TIGIT and CD112R in some aspects are markedly overexpressed by modified immune cells that were engineered to target an antigen (e.g., tumor-associated antigen). In some embodiments, such overexpression is observed following, but not before, adoptive transfer of the modified immune cells to a subject with a tumor such as a solid tumor, such as a tumor associated with a common epithelial cancer. This overexpression in some aspects is observed to correlate with functional impairment of the modified immune cells' antitumor activity or function.

Several solid tumor antigens have been identified as potential therapeutic targets. See, e.g., Xia et al., Oncotarget (ISSN:1949-2553, 2017). An exemplary solid tumor antigen is the receptor tyrosine kinase ROR1. Briefly, ROR1 is an oncofetal antigen that is overexpressed in a wide variety of tumors, yet is expressed in few normal tissues. ROR1 is highly expressed in certain solid cancers, including epithelial cancers, and in B-cell chronic lymphocytic leukemia (CLL) and mantle cell lymphoma (MCL). Based on high expression of ROR1 on the cell surface of tumors and minimal ROR1 expression in normal tissues, ROR1 is a favorable tumor-specific or tumor-associated antigen to target with therapeutics. For example, T cells expressing a chimeric antigen receptor (CAR) have been designed to target ROR1-expressing tumors (see Hudecek et al., Blood 116: 4532-41, 2010; Hudecek et al., Clin. Cancer Res. 19(12):3153 (2013); Berger et al., Cancer Immunol. Res. 3(2):206 (2015), and PCT Publication No. WO 2014/031687, the ROR1-specific CAR constructs (including the binding domains, extracellular domains (including spacers), transmembrane domains, intracellular domains, costimulatory domains, transduction markers, and related amino acid and nucleotide sequences) of which are incorporated herein by reference in their entireties).

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “nucleic acid” or “nucleic acid molecule” refers to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. Nucleic acids may be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded. A “polynucleotide” refers to a polymeric compound including covalently linked nucleotides, which can be made up of natural subunits (e.g., purine or pyrimidine bases) or non-natural subunits (e.g., morpholine ring).

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

The terms “treat” and “treatment” refer to medical management of a disease, disorder, or condition of a subject (i.e., patient, host, who may be a human or non-human animal) (see, e.g., Stedman's Medical Dictionary). In general, an appropriate dose and treatment regimen according to the methods and compositions described herein results in a therapeutic or prophylactic benefit. Therapeutic or prophylactic benefit resulting from therapeutic treatment or prophylactic or preventative methods include, for example, an improved clinical outcome, wherein the object is to prevent or retard or otherwise reduce (e.g., decrease in a statistically significant manner relative to an untreated control) an undesired physiological change or disorder, or to prevent, retard or otherwise reduce the expansion or severity of such a disease or disorder. Beneficial or desired clinical results from treating a subject include abatement, lessening, or alleviation of symptoms that result from or are associated with the disease or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; or overall survival.

“Treatment” can also mean prolonging survival when compared to expected survival if a subject were not receiving treatment. Subjects in need of the methods and compositions described herein include those who already have the disease or disorder, as well as subjects prone to have or at risk of developing the disease or disorder. Subjects in need of prophylactic treatment include subjects in whom the disease, condition, or disorder is to be prevented (i.e., decreasing the likelihood of occurrence or recurrence of the disease or disorder). The clinical benefit provided by the compositions (and preparations comprising the compositions) and methods described herein can be evaluated by design and execution of in vitro assays, preclinical studies, and clinical studies in subjects to whom administration of the compositions is intended to benefit, as described in the examples.

A “patient” or “subject” includes an animal, such as a human, cow, horse, sheep, lamb, pig, chicken, turkey, quail, cat, dog, mouse, rat, rabbit or guinea pig. The animal can be a mammal, such as a non-primate or a primate (e.g., monkey, ape, and human). In some embodiments, a patient is a human, such as a human infant, child, adolescent, or adult.

As used herein, “administration” of a composition or therapy refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with of multiple agents may include simultaneous, concurrent, or sequential delivery of the agents in any order and on any dosing schedule.

“Effective amount” or “therapeutically effective amount” refers to that amount of a composition described herein which, when administered to a mammal (e.g., human), is sufficient to aid in treating a disease. The amount of a composition that constitutes an “effective amount” will vary depending on the cell preparations, the condition and its severity, the manner of administration, and the age of the mammal to be treated, but can be determined routinely by one of ordinary skill in the art having regard to his own knowledge and to this disclosure. When referring to an individual active ingredient or composition, administered alone, an effective dose refers to that ingredient or composition alone. When referring to a combination, an effective dose refers to combined amounts of the active ingredients, compositions or both that result in the therapeutic effect, whether administered serially, concurrently or simultaneously.

In general, a therapeutically effective amount of a composition or combination of agents according to this disclosure results in an improvement in an antitumor functionality of a modified immune cell as described herein. Non-limiting examples of antitumor functionalities include cytotoxic or cytolytic activity by the modified immune cell against the tumor, proliferation of the modified immune cell, persistence of the modified immune cell (or functional progeny thereof) over time, localization of the modified immune cell to a tumor site, and the production and release of pro-inflammatory cytokines by the modified immune cell in response to stimulation or re-stimulation with a tumor-associated antigen (e.g., by a CAR-expressing T cell specific for ROR1 antigen in response to stimulation or restimulation with ROR1). An improvement in an antitumor functionality can be determined by comparing the functionality in a modified immune cell according to the present disclosure with that of a reference cell (i.e., with that of a modified or unmodified immune cell in which TIGIT or CD112R expression or activity is not inhibited, such as a reference immune cell that is capable of specifically binding to a same tumor-associated antigen as the modified immune cell in which TIGIT or CD112R expression or activity is inhibited), or by comparing the functionality of the modified immune cell before and after an intervention to inhibit TIGIT or CD112R expression or activity therein (e.g., by administration of an agent that inhibits TIGIT or CD112R expression or activity).

As used herein, an “immune cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and natural killer T (NK-T cells)). Exemplary immune system cells include a CD4⁺ T cell, a CD8⁺ T cell, a CD4⁻ CD8⁻ double negative T cell, a γδ T cell, a regulatory T cell, a natural killer (NK) cell, a NK-T cell (also called a cytokine-induced killer cell or CIK cell), and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune cell that matures in the thymus and produces T cell receptors (TCRs). T cells can be naïve (not exposed to antigen; increased expression of CD62L, CCR7, CD28, CD3ζ, CD127, and CD45RA, and decreased expression of CD45RO as compared to T_{CM}), memory T cells (T_{M}) (antigen-experienced and long-lived), including stem cell memory T cells, and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}). Effector T cells (T_{E}) refers to antigen-experienced CD8+ cytotoxic T lymphocytes that have decreased expression of CD62L, CCR7, CD28, and are positive for granzyme and perforin as compared to T_{CM}. Other exemplary T cells include regulatory T cells, such as CD4⁺ CD25⁺ (Foxp3⁺) regulatory T cells and Treg17 cells, as well as Tr1, Th3, CD8⁺ CD28⁻, and Qa-1 restricted T cells. In certain embodiments of the present disclosure, a modified immune cell comprises a T cell, a Treg cell, a NK cell, a NK-T cell, or any combination thereof. In further embodiments, the modified immune cell is a CD4⁺ T cell and/or a CD8⁺ T cell.

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻ CD117⁺ phenotype or those found in the thymus (referred to as progenitor thymocytes).

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein, or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

As used herein, the term “modified” refers to a cell, microorganism, nucleic acid molecule, or vector that has been genetically engineered by human intervention—e.g., a cell that has been modified by introduction of a heterologous polynucleotide (or a progeny cell thereof), or refers to a cell or microorganism that has been altered such that expression of an endogenous polynucleotide or gene is controlled, deregulated, abrogated, or constitutive (or the progeny of such a cell or microorganism that comprises such an alteration compared to a reference un-altered cell or microorganism). Human-generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

The term “heterologous” polynucleotide, construct or sequence refers to a nucleic acid molecule or portion of a polynucleotide that is not native to a host cell, but may be homologous to a polynucleotide or portion of a polynucleotide from the host cell. The source of the heterologous polynucleotide, construct or sequence may be from a different genus or species. In certain embodiments, a heterologous polynucleotide is added (i.e., not endogenous or native) to a host cell or host genome by, for example, conjugation, transformation, transfection, electroporation, gene-editing, homologous recombination, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein, or other activity-associated biomolecule encoded by a heterologous polynucleotide introduced into the host cell, even if the host cell encodes a same homologous protein, enzyme, or other activity-associated biomolecule.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous nucleic acid molecules encoding a desired binding receptor. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

The term “operably linked” or “operatively associated” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “introduced” in the context of inserting a nucleic acid molecule into a cell, means “transfection,” “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). The term “homologous” or “homolog” refers to a molecule or activity found in or derived from a host cell, species or strain. For example, a heterologous polynucleotide may be homologous to a native host cell gene, and may optionally have an altered expression level, a different sequence, an altered activity, or any combination thereof.

### Antigen-Specific Receptors

In certain aspects, the present disclosure provides antigen-specific receptors, such as may be expressed by a modified immune cell to target a disease (e.g., tumor)-associated antigen. The term “antigen-specific receptor” as used herein, refers to a naturally occurring, modified, or synthetic receptor protein that is expressed at the cell surface (e.g., heterologously or endogenously) and comprises a extracellular component comprising a binding domain that specifically binds to a cognate antigen, a hydrophobic or transmembrane component, and an intracellular signaling component that, upon binding of the antigen by the binding domain, initiates a signal to the cell to elicit a cellular response (e.g., a growth or proliferation signal, or a signal to produce and release cytokines). Examples of antigen-specific receptors include CARs and TCRs, as described herein.

Exemplary antigens that can be bound by antigen-specific receptors of the present disclosure include ROR1, EGFR, EGFRvIII, EGP-2, EGP-40, GD2, GD3, HPV E6, HPV E7, HER2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, MAGE-A3, MAGE-A4, PRAME, PSA, ephrin A2, ephrin B2, an NKG2D, NY-ESO-1, TAG-72, mesothelin, NY-ESO, SSX-2, SSX-3, 5T4, BCMA, FAP, Carbonic anhydrase 9, ERBB2, BRAF^{V600E}, and CEA. Antigens of this disclose include those that are presented by an MHC complex; e.g., an HLA complex. An antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” or includes any molecule, structure, amino acid sequence or protein determinant that is recognized and specifically bound by a cognate binding molecule, such as an immunoglobulin (e.g., antibody), T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

A receptor binding domain (also referred to as a “binding region” or “binding moiety”), as used herein, refers to a molecule or portion thereof that possesses the ability to specifically and non-covalently associate, unite, or combine with a target (e.g., a tumor-associated antigen). A binding domain includes any naturally occurring, synthetic, semi-synthetic, or recombinantly produced binding partner for a biological molecule, a molecular complex (i.e., complex comprising two or more biological molecules), or other target of interest. For example, a binding domain may comprise a natural antibody, a synthetic or recombinant antibody construct, or a binding fragment thereof. For example, a binding domain may comprise a full-length antibody heavy chain or light chain, a Fab fragment, a Fab′, a F(ab)₂, a heavy chain variable domain (VH domain), a light chain variable domain (VL domain), a domain antibody (dAb), a single domain camelid antibody (VHH), a heavy chain-only antibody (e.g., an IgNAR from a cartilaginous fish) a complementary determining region (CDR) or a binding fragment thereof, a single chain variable fragment (scFv), or the like. Other examples of binding domains include those from (or derived from) T cell receptors (TCRs); e.g., TCR variable domains (including those from single chain T cell receptors), extracellular domains of receptors, receptor ectodomains, ligands for cell surface receptors/molecules, tumor binding proteins/peptides, cytokines, chemokines, or synthetic polypeptides selected for their specific ability to bind to a biological molecule, a molecular complex or other target of interest. In certain embodiments, a receptor binding domain is murine, camelid, from a cartilaginous fish (e.g., a shark, ray, or skate), chimeric, human, or humanized. In certain embodiments, an antigen-specific receptor comprises a binding domain from (or is) an antibody, a TCR or a CAR.

“TCR” refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). The extracellular portion of a TCR chain (e.g., α-chain, β-chain), like an antibody monomer, comprises two immunoglobulin domains: a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminus; and a constant domain (e.g., α-chain constant domain or C_{α}, typically amino acids 117 to 259 based on Kabat, β-chain constant domain or C_{β}, typically amino acids 117 to 295 based on Kabat) adjacent to the cell membrane. Also like antibodies, the variable domains of a TCR contain complementary determining regions (CDRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, rabbit or other mammal. Single chain TCR constructs (scTCRs), which comprise a single constant domain and an associated variable domain (e.g. a TCR Vα) linked (e.g., by a peptide linker) to a cognate variable domain (e.g. a TCR Vβ), are also contemplated herein.

The term “variable region” or “variable domain”, as applied to an immunoglobulin binding protein, such an antibody or a TCR (or a binding fragment thereof), refers to the domain of an antibody heavy chain or light chain, or a TCR α-chain or β-chain (or γ chain and δ chain for γδ TCRs) that is involved in binding of the immunoglobulin binding protein to antigen. The variable domains of immunoglobulin binding proteins generally have similar structures, with each domain comprising four conserved framework regions (FRs) and three complementarity determining regions (CDRs). The terms “complementarity determining region” (CDR) or “hypervariable region” (HVR) are known in the art to refer to non-contiguous sequences of amino acids within antibody variable regions, which confer antigen specificity or binding affinity. In general, there are three CDRs in each heavy chain variable region (HCDR1, HCDR2, and HCDR3) and three CDRs in each light chain variable region (LCDR1, LCDR2, and LCDR3).

“Framework regions” (FR), as used herein, refer to the non-CDR portions of the variable regions of immunoglobulin binding proteins. In general, there are four FRs in each full-length heavy or light chain variable region of an antibody (or in each full-length α, β, γ, or δ-chain variable region of a TCR).

In general, Vα domain (of TCRs) or V_{L} domain (of antibodies) is encoded by two separate DNA segments, the variable gene segment and the joining gene segment (V-J); the Vβ domain of TCRs (or the V_{H} of antibodies) is encoded by three separate DNA segments, the variable gene segment, the diversity gene segment, and the joining gene segment (V-D-J). A single variable domain may be sufficient to confer antigen-binding specificity. Furthermore, variable domains that bind ti a particular antigen may be isolated using a variable domain from an immunoglobulin binding protein that binds the antigen to screen a library of complementary variable domains, respectively.

“CD3” is known in the art as a multi-protein complex of six chains (see, Abbas and Lichtman, 2003; Janeway et al., p. 172 and 178, 1999) that are involved in antigen-specific signaling in T cells. In mammals, the complex comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is is thought to allow these chains to associate with positively charged regions of T cell receptor chains. The intracellular tails of the CD3γ, CD3δ, and CD3ε chains each contain a single conserved motif known as an immunoreceptor tyrosine-based activation motif or ITAM, whereas each CD3 chain has three ITAMs. CD3 has a short ectodomain (extracellular domain), a transmembrane domain, and an intracellular domain, and typically forms a homodimer in a TCR complex. Without wishing to be bound by theory, it is believed that ITAMs are important for the signaling capacity of a TCR complex. CD3 as used in the present disclosure may be from various animal species, including human, mouse, rat, or other mammals.

As used herein, “TCR complex” refers to a complex formed by the association of CD3 proteins with a TCR. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCR chain.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Major histocompatibility complex” (MHC) refers to glycoproteins that deliver peptide antigens to a cell surface. MHC class I molecules are heterodimers having a membrane spanning a chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. Human MHC is referred to as human leukocyte antigen (HLA); e.g., of HLA-I or HLA-II subtype. HLA-II types include DP, DM, DOA, DOB, DQ, and DR. Numerous alleles encoding the subunits of the various HLA types are known, including, for example, HLA-DQA1*03, HLA-DQB1*0301, HLA-DQB1*0302, HLA-DQB1*0303.

“CD8” is an immunoglobulin co-receptor glycoprotein that assists the TCR in communicating with antigen-presenting cells expressing MHC Class I receptors (see, Campbell & Reece, Biology 911 (Benjamin Cummings, Sixth Ed., 2002)). CD8 is found on the surface of immune cells such as cytotoxic T cells, natural killer cells, cortical thymocytes, and dendritic cells, and typically includes exists as a heterodimer comprised of two chains (CD8α and CD8β), though it can also exist as an alpha-alpha homodimer. In humans, five (5) different CD8 beta chain isoforms (see UniProtKB identifier P10966) and a single CD8 alpha chain isoform (see UniProtKB identifier P01732) are known, though variants of these also exist.

Each chain of a naturally occurring CD8 dimer molecule possesses an immunoglobulin variable-like extracellular domain, a thin stalk, and a short cytoplasmic tail. During antigen presentation, CD8 is recruited to bind to the α3 portion of the MHCI molecule. Without wishing to be bound by theory, it is believed that upon antigen binding, the CD8 intracellular tails interact with Lck (lymphocyte-specific protein tyrosine kinase), which phosphorylates the CD3 and TCRζ chains to send signals to the T cell.

“CD4” is an immunoglobulin co-receptor glycoprotein that assists the TCR in communicating with antigen-presenting cells expressing MHC Class II receptors (see, Campbell & Reece, Biology 909 (Benjamin Cummings, Sixth Ed., 2002)). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII β2, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce various types of T helper cells.

As used herein, the term “chimeric antigen receptor” (CAR) refers to a fusion protein comprising two or more naturally occurring amino acid sequences or portions thereof linked together in a way that does not occur naturally or does not occur naturally in a host cell, which fusion protein can function as a receptor when present on the surface of a cell and comprises an extracellular component comprising an antigen-binding domain specific for an antigen (e.g., obtained or derived from an immunoglobulin molecule, such as a scFv or scTCR derived from an antibody or TCR specific for a cancer antigen, or an antigen-binding domain derived or obtained from a killer immunoreceptor from an NK cell), a hydrophobic portion or transmembrane domain, and an intracellular component that, in some embodiments, is capable of activating or stimulating an immune cell, and optionally comprises a spacer located between the binding domain and the transmembrane domain of the CAR. In certain embodiments, a spacer region provides flexibility for the binding domain to efficiently bind a target or ligand, allow for high expression levels in immune cells, or both.

A hydrophobic portion or transmembrane domain is typically disposed between the extracellular antigen-binding domain and the intracellular signaling component, and transverses and anchors the CAR in a host cell membrane (e.g., T cell). A chimeric antigen receptor may further comprise an extracellular spacer domain connecting the hydrophobic portion or transmembrane domain and the extracellular antigen binding domain.

An intracellular component of an antigen-specific receptor, such as a CAR, may be from a T cell or other receptor or portion thereof, such as an intracellular activation domain (e.g., an immunoreceptor tyrosine-based activation motif (ITAM)-containing T cell activating motif), an intracellular costimulatory domain, or both. In certain embodiments, an intracellular component of a CAR of this disclosure comprises an effector domain. As used herein, an “effector domain” is an intracellular portion or domain of an antigen-specific receptor (e.g., a CAR) that can directly or indirectly promote a biological or physiological response in a cell when receiving an appropriate signal. In certain embodiments, an effector domain is from a protein or portion thereof or protein complex that receives a signal when bound, or when the protein or portion thereof or protein complex binds directly to a target molecule and triggers a signal from the effector domain.

An effector domain may directly promote a cellular response when it contains one or more signaling domains or motifs, such as an Intracellular Tyrosine-based Activation Motif (ITAM), such as those found in costimulatory molecules. In certain embodiments, the intracellular component or functional portion thereof comprises an ITAM. Exemplary effector domains include those from, CD3ε, CD3δ, CD3ζ, CD25, CD79A, CD79B, CD5, CD22, CD38, CD66D, CARD11, DAP10, FcRα, FcRβ, FcγR, Fyn, HVEM, ICOS, Lck, LAG3, LAT, LRP, NKG2D, CD94, NOTCH1, NOTCH2, NOTCH3, NOTCH4, Wnt, ROR2, Ryk, SLAMF1, Slp76, pTa, TCRα, TCRβ, TRIM, Zap70, PTCH2, or any combination thereof. In certain embodiments, an effector domain comprises a lymphocyte receptor signaling domain (e.g., CD3ζ) or a functional portion or variant thereof.

In further embodiments, the intracellular component of the antigen-specific receptor (e.g., CAR) comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), CD7, GITR, CD30, CD40, BAFF-R, HVEM, LIGHT, NKG2C, SLAMF7, NKp80, CD160, B7-H3, a ligand that specifically binds with CD83, or a functional variant thereof, or any combination thereof. In certain embodiments, the intracellular component comprises a CD28 costimulatory domain or a functional portion or variant thereof (which may optionally include a LL→GG mutation at positions 186-187 of the native CD28 protein (see Nguyen et al., Blood 102:4320, 2003)), a 4-1BB costimulatory domain or a functional portion or variant thereof, or both.

Methods of making CARs and CAR designs are known; see, e.g., U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; PCT Publication No. WO2014/134165; and Brentjens et al., 2007, Clin. Cancer Res. 13:5426; Sadelain et al., Cancer Discov., 3(4):388 (2013); see also Harris and Kranz, Trends Pharmacol. Sci., 37(3):220 (2016); Stone et al., Cancer Immunol. Immunother., 63(11):1163 (2014), which methods and CAR designs are incorporated by reference herein. In certain embodiments, a binding protein comprises a CAR comprising an antigen-specific binding domain from a TCR (see, e.g., Walseng et al., Scientific Reports 7:10713, 2017; the TCR CAR constructs and methods of which are incorporated by reference herein).

Exemplary CARs may have two or more portions from the same protein linked in a way not normally found in a cell, or a CAR may have portions from two, three, four, five or more different proteins linked in a way not normally found in a cell. Furthermore, CARs can be in the form of first, second or third generation CARs. For example, a first generation CAR generally may have a single intracellular signaling domain providing an activation signal (e.g., intracellular signaling domain of CD3 or FcγRI or other ITAM-containing domain). Second generation CARs further include an intracellular costimulatory domain (e.g., a costimulatory domain from an endogenous T cell costimulatory receptor, such as CD28, 4-1BB, or ICOS). Third generation CARs further include a second costimulatory domain. In certain embodiments, a CAR comprises at least one co-stimulatory domain.

Binding of an antigen-specific receptor protein of the present disclosure (e.g., expressed on the surface of a modified immune cell as disclosed herein) to a target antigen can be determined using any number of assays that are known in the art. “MHC-peptide tetramer staining” refers to an assay used to detect antigen-specific T cells, which features a tetramer of MHC molecules, each comprising an identical peptide having an amino acid sequence that is cognate (e.g., identical or related to) at least one antigen, wherein the complex is capable of binding T cell receptors specific for the cognate antigen. Each of the MHC molecules may be tagged with a biotin molecule. Biotinylated MHC/peptides are tetramerized by the addition of streptavidin, which can be fluorescently labeled. The tetramer may be detected by flow cytometry via the fluorescent label. In certain embodiments, an MHC-peptide tetramer assay is used to detect or select enhanced affinity TCRs of the instant disclosure.

As used herein, “specifically binds” or “specific for” refers to an association or union of a binding protein (e.g., CAR or TCR) or a binding component (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding proteins or binding domains (or fusion proteins thereof) may be classified as “high affinity” binding proteins or binding domains (or fusion proteins thereof) or as “low affinity” binding proteins or binding domains (or fusion proteins thereof). “High affinity” binding proteins or binding domains refer to those binding proteins or binding domains having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding proteins or binding domains refer to those binding proteins or binding domains having a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof.

In certain embodiments, a binding protein of this disclosure may be codon optimized to enhance expression in a particular host cell, such as T cells (Scholten et al., Clin. Immunol. 119:135, 2006).

A variety of assays can be used to identify binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). In some examples, apparent K_{D} of a TCR is measured using 2-fold dilutions of labeled tetramers at a range of concentrations, followed by determination of binding curves by non-linear regression, apparent K_{D} being determined as the concentration of ligand that yielded half-maximal binding.

Antigen-specific receptors expressed by modified immune cells according to the present disclosure specifically bind to antigens that are associated with, for example, solid tumors. Solid tumors are typically associated with certain hyperproliferative disorders. As used herein, “hyperproliferative disorder” refers to excessive growth or proliferation as compared to a normal or undiseased cell. Exemplary hyperproliferative disorders that produce solid tumors include certain cancers, neoplastic tissue, carcinoma, sarcoma, pre-malignant cells, as well as non-neoplastic or non-malignant hyperproliferative disorders (e.g., adenoma, fibroma, lipoma, leiomyoma, hemangioma, restenosis, as well as autoimmune diseases such as rheumatoid arthritis, osteoarthritis, psoriasis, inflammatory bowel disease, or the like).

### Modified Immune Cells

In further aspects, the present disclosure provides a genetically modified immune cell comprising a heterologous polynucleotide encoding an antigen-specific receptor that specifically binds to a tumor-associated antigen. In certain embodiments, an immune cell is a T cell (e.g., a CD4⁺ T cell, a CD8⁺ T cell, a stem cell memory T cell, or the like), a Treg cell, a NK cell, or an NK-T cell, or any combination thereof. In certain embodiments, an immune cell is a CD4⁺ T cell or a CD8+ T cell. In further embodiments, an immune cell is a human immune cell. It will be understood that a modified immune cell encompasses the progeny of a modified immune cell; e.g., a parent immune cell may be modified to comprise a heterologous polynucleotide encoding an antigen-specific receptor, and may subsequently divide to produce a progeny cell that comprises the polynucleotide—both the parent and the progeny cell are “modified immune cells” according to the present disclosure.

In further embodiments, an encoded antigen-specific receptor can comprise a T cell receptor (TCR) or a chimeric antigen receptor (CAR).

In certain embodiments, the antigen-specific receptor comprises a TCR. In certain embodiments, a TCR comprises an enhanced affinity TCR. In certain embodiments, the TCR is an αβ TCR or a γδ TCR.

In certain embodiments, an antigen-specific receptor comprises a CAR.

In certain embodiments, a modified immune cell of this disclosure binds, via an antigen-specific receptor, to a tumor-associated antigen. In some embodiments, the tumor-associated antigen is selected from or comprises ROR1, EGFR, EGFRvIII, EGP-2, EGP-40, GD2, GD3, HPV E6, HPV E7, HER2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, PSA, ephrin A2, ephrin B2, an NKG2D, NY-ESO-1, SSX-2, SSX-3, TAG-72, mesothelin, NY-ESO, 5T4, BCMA, FAP, Carbonic anhydrase 9, ERBB2, BRAE^{V600E}, or CEA.

In certain embodiments, the tumor-associated antigen is ROR1. Any of a number of antibodies specific for ROR1 and the variable regions or CDRs thereof can be readily used to make antigen-specific receptor (e.g., CAR) constructs of this disclosure. Incorporated herein by reference are all of the ROR1 antibodies and related protein and nucleic acid constructs and related variable domain, Fab, and CDR sequences disclosed in WO 2014/031687; WO 2012/076066; US 2015/0232569; US 2012/0058051; U.S. Pat. No. 9,316,646; US 2013/0251642; U.S. Pat. No. 9,217,040; Yang et al., PLoS One 6(6):e21018 (2011); and Choi et al., Clin. Lymphoma Myeloma Leu. (2015) 15(Suppl):5167-5169. In particular embodiments, the antigen-specific receptor comprises a binding domain derived from antibody 2A2, antibody R12, antibody R11, antibody Y31, antibody UC-961, antibody D10, or antibody H10. In some embodiments, the antigen-specific receptor binding domain derived from antibody 2A2, antibody R12, antibody R11, antibody Y31, antibody UC-961, antibody D10, or antibody H10 has a VH or (i.e., and/or) a VL having at least about 80%, 85%, 90%, 95%, 96%, 96%, 98%, 99%, or more amino acid sequence identity to that of the antibody variable regions or scFv thereof from antibody R12, antibody 2A2, antibody R12, antibody R11, antibody Y31, antibody UC-961, antibody D10, or antibody H10.

In certain embodiments, a binding domain of an antigen-specific receptor comprises HCDRs according to SEQ ID NOs:1-3 and LCDRs according to SEQ ID NOs:4-6.

In other embodiments, a binding domain of an antigen-specific receptor comprises HCDRs according to SEQ ID NOs:10-12 and LCDRs according to SEQ ID NOs:13-15.

In still other embodiments, a binding domain of an antigen-specific receptor comprises HCDRs according to SEQ ID NOs:19-21 and LCDRs according to SEQ ID NOs:22-24.

In other embodiments, a binding domain of an antigen-specific receptor comprises HCDRs according to SEQ ID NOs:28-30 and LCDRs according to SEQ ID NOs:31-33.

In yet other embodiments, a binding domain of an antigen-specific receptor comprises HCDRs according to SEQ ID NOs:34-37 and LCDRs according to SEQ ID NOs:38-40.

In still other embodiments, a binding domain of an antigen-specific receptor comprises HCDRs according to SEQ ID NOs:40-42 and LCDRs according to SEQ ID NOs:43-45.

In still further embodiments, antibody variable regions of a binding domain (e.g., a scFv of a CAR molecule) of this disclosure have at least 90% amino acid sequence identity to that of the antibody variable regions or scFv thereof from antibody R12, antibody 2A2, antibody R12, antibody R11, antibody Y31, antibody UC-961, antibody D10, or antibody H10.

In some embodiments, the binding domain includes a heavy chain variable domain (VH) comprising or consisting of an amino acid sequence having at least 80%, 85%, 90%, 91%, 92%, 93%, 94%, 95%, 96%, 97%, 98%, 99%, or 100% sequence identity the amino acid sequence set forth in SEQ ID NO:7, and a light chain variable domain heavy chain variable domain (VL) comprising or consisting of an amino acid sequence having at least 80%, 85%, 90%, 91%, 92%, 93%, 94%, 95%, 96%, 97%, 98%, 99%, or 100% sequence identity to amino acid sequence set forth in SEQ ID NO:8.

In other embodiments, the binding domain comprises a VH comprising or consisting of an amino acid sequence having at least 80%, 85%, 90%, 91%, 92%, 93%, 94%, 95%, 96%, 97%, 98%, 99%, or 100% sequence identity the amino acid sequence set forth in SEQ ID NO:16, and a VL comprising or consisting of an amino acid sequence having at least 80%, 85%, 90%, 91%, 92%, 93%, 94%, 95%, 96%, 97%, 98%, 99%, or 100% sequence identity the amino acid sequence set forth in SEQ ID NO:17.

In other embodiments, the binding domain comprises a VH comprising or consisting of an amino acid sequence having at least 80%, 85%, 90%, 91%, 92%, 93%, 94%, 95%, 96%, 97%, 98%, 99%, or 100% sequence identity the amino acid sequence set forth in SEQ ID NO:25, and a VL comprising or consisting of an amino acid sequence having at least 80%, 85%, 90%, 91%, 92%, 93%, 94%, 95%, 96%, 97%, 98%, 99%, or 100% sequence identity the amino acid sequence set forth in SEQ ID NO:26.

In particular embodiments, a ROR1-specific CAR includes a scFv binding domain derived from anti-ROR1 antibody R12, antibody 2A2, antibody R11, antibody Y31, antibody UC-961, antibody D10, or antibody H10, which scFv binding domain can, in certain embodiments, be human or humanized. Any scFv of the present disclosure may be engineered so that the C-terminal end of the V_{L} domain is linked by a short peptide sequence to the N-terminal end of the V_{H} domain, or vice versa (i.e., (N)V_{L}(C)-linker-(N)V_{H}(C) or (N)V_{H}(C)-linker-(N)V_{L}(C).

In certain embodiments, a ROR1-specific scFv comprises or consists of the amino acid sequence set forth in SEQ ID NO:9. In other embodiments, a ROR1-specific scFv comprises or consists of the amino acid sequence set forth in SEQ ID NO:18. In still other embodiments, a ROR1-specific scFv comprises or consists of the amino acid sequence set forth in SEQ ID NO:27.

In any of the aforementioned embodiments, a ROR1-specific CAR of this disclosure has a short or intermediate spacer of about 120 amino acids or less, about 100 amino acids or less, about 75 amino acids or less, about 50 amino acids or less, about 20 amino acids or less, about 15 amino acids or less, about 12 amino acids or less, or about 10 amino acids or less. Incorporated by reference herein are all of the CAR spacers disclosed in PCT Publication No. WO 2014/031687, including the spacer amino acid sequences and spacer lengths. In certain embodiments, a ROR1-specific CAR comprises a 12-amino acid spacer having the amino acid sequence of ESKYGPPCPPCP (SEQ ID NO:47) or ESKYGPPCPSCP (SEQ ID NO:48).

Construction of an expression vector to produce an antigen-specific receptor of the instant disclosure in a host cell (e.g., an immune cell) can be accomplished by using any suitable molecular biology engineering techniques, including the use of restriction endonuclease digestion, ligation, transformation, plasmid purification, and DNA sequencing as described in, for example, Sambrook et al. (1989 and 2001 editions; Molecular Cloning: A Laboratory Manual, Cold Spring Harbor Laboratory Press, NY) and Ausubel et al. (Current Protocols in Molecular Biology, 2003). To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the immunogen. Methods for transfecting/transducing immune cells with desired polynucleotides have been described (e.g., U.S. Patent Application Pub. No. US 2004/0087025) as have adoptive transfer procedures using immune cells of desired antigen-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), which methods are hereby incorporated herein by reference.

In certain embodiments, a construct comprising a heterologous polynucleotide encoding an antigen-specific receptor that specifically binds to a tumor-associated antigen is comprised in a viral vector. Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

Antigen-specific receptors as described herein and modified immune cells expressing the antigen-specific receptors may be functionally characterized according to any of a large number of methodologies for assaying immune cell activity, including, for example, determination of T cell binding, activation or induction and also including determination of T cell responses that are antigen-specific. Examples include determination of T cell proliferation, T cell cytokine release, antigen-specific T cell stimulation, MHC-restricted T cell stimulation, CTL activity (e.g., by detecting ⁵¹Cr release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein.

Levels of cytokines may be determined according to methods described herein, including for example, ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

In another aspect, the present disclosure provides a genetically modified immune cell, comprising (a) a heterologous polynucleotide encoding an antigen-specific receptor (e.g., CAR or TCR) that specifically binds to a tumor-associated antigen, and (b) a chromosomal TIGIT gene knockout or mutation or a chromosomal CD112R gene knockout or mutation. Additionally or alternatively, a modified immune cell can comprise a chromosomal gene knockout or mutation of one or more of: PD-1; LAG-3; CTLA-4; TIM3; an HLA molecule; a TCR molecule, or any component or combination thereof.

As disclosed herein, TIGIT, CD112R, and other endogenously expressed immune cell proteins (e.g., PD-1, LAG-3, CTLA4, TIM3) can inhibit or reduce the immune activity of a modified immune host cell, or may compete (e.g., TCR) with an antigen-specific binding protein of the present disclosure for expression by the host cell, or may interfere with the binding activity of a heterologously expressed antigen-specific binding protein of the present disclosure and interfere with the modified immune cell binding to a target cell or antigen. Further, endogenous proteins (e.g., endogenous host cell proteins, such as an HLA) expressed by a donor immune cell to be used in a cell transfer therapy may be recognized as foreign by an allogeneic recipient, which may result in elimination or suppression of the donor immune cell by the allogeneic recipient, or can render the administered donor cell to be immunogenic in an allogeneic recipient.

Accordingly, decreasing or eliminating expression or activity of such endogenous genes or proteins can improve the activity, function, expansion or persistence, or reduce the risk of immunogenicity, of the administered host cells in an autologous or allogeneic recipient, and can allow universal administration of the modified host cells (e.g., to any recipient regardless of HLA type). In certain embodiments, a modified host immune cell is an allogeneic or an autologous cell.

In some embodiments, the term “chromosomal gene knockout” or mutation refers to a genetic alteration in a host cell that prevents or inhibits production, by the host cell, of a functionally active endogenous polypeptide product. In some aspects, the modified host immune cell is modified to introduce a mutation, such as a deletion, insertion, substitution, missense mutation and/or nonsense mutation, at one or more of a gene that encodes TIGIT, CD112R, PD-1, LAG-3, CTLA4, TIM3, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region). Alterations resulting in a chromosomal gene knockout or a mutation can include, for example, introduced nonsense mutations (including the formation of premature stop codons), missense mutations, gene deletion, and strand breaks. In some aspects, decreasing or eliminating expression or activity of such endogenous genes or proteins can be carried out by heterologous expression of inhibitory nucleic acid molecules that inhibit endogenous gene expression in the host cell. In some aspects, modifications can introduce nucleic acid sequences that are different from the endogenous nucleic acid sequence at the endogenous genes encoding such proteins, for example, by knock-in of a different sequence at the endogenous genes.

In certain embodiments, a modified host immune cell of this disclosure comprises a chromosomal gene knockout or introduction of a mutation, for example, using gene editing, of one or more of a gene that encodes TIGIT, CD112R, PD-1, LAG-3, CTLA4, TIM3, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region), or any combination thereof. In certain embodiments, a chromosomal gene knock-out, gene knock-in or mutation is introduced by chromosomal editing of a host cell, e.g., using gene editing methods (see, e.g., Torikai et al., Nature Sci. Rep. 6:21757 (2016); Torikai et al., Blood 119(24):5697 (2012); and Torikai et al., Blood 122(8): 1341 (2013); the gene-editing methods, techniques, compositions, and adoptive cell therapies of which are herein incorporated by reference in their entirety). For example, in some embodiments, a chromosomal gene knockout is produced using a CRISPR/Cas9 system, and may involve transfection of the modified immune cell with a lentivirus (e.g., pLentiCRISPRv2; Torikai et al., Blood (2016)) expressing a CRISPR/Cas9 system targeting PD-1, LAG-3, CTLA4, an HLA component, or a TCR component, or any combination thereof. Primers useful for designing a lentivirus that expresses a CRISPR/Cas9 system for inhibiting an endogenously expressed immune cell protein include for example, primer pairs comprising forward and reverse primers having the nucleotide sequences set forth in SEQ ID NOS:58 and 59, 60 and 61, 62 and 63, and 64 and 65.

Chromosomal editing or gene editing can be performed using, for example, endonucleases, such as targeted endonucleases. In some aspects, “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event, and/or by introduction of particular mutations, such as a deletion, insertion, substitution, missense mutation and/or nonsense mutation, at the targeted gene. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases, such as targeted endonucleases, include zinc finger nucleases, TALE-nucleases, CRISPR-Cas nucleases, meganucleases, and megaTALs.

In some embodiments, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

In some embodiments, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” in some aspects is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

In some embodiments, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al. (Clin. Cancer Res. 23:2255-2266, (2017), the gRNAs, Cas9 DNAs, vectors, and gene knockout techniques of which are hereby incorporated by reference in their entirety.

In some embodiments, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-Pspl, PI-Sce, I-SceIV, I-Csml, I-PanI, I-SceII, I-PpoI, I-SceIII, I-CreI, I-Teel, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from TIGIT, CD112R, PD-1, LAG3, TIM3, CTLA4, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, the host cell (e.g., an immune cell) comprising a heterologous polynucleotide encoding a binding protein that specifically binds to a disease-associated antigen can be modified to inhibit, reduce or eliminate expression of one or more endogenous genes by introducing an inhibitory nucleic acid molecule into the cell. In some aspects, the inhibitory nucleic acid molecule encodes a target-specific inhibitor wherein the encoded target-specific inhibitor inhibits endogenous gene expression (e.g., of TIGIT, CD112R, PD-1, TIM3, LAG3, CTLA4, an HLA component, or a TCR component, or any combination thereof) in the host immune cell.

The presence of the introduced chromosomal gene knockout or mutation can be confirmed directly by DNA sequencing of the host immune cell following use of the knockout procedure or agent. Chromosomal gene knockout, mutation, or inhibition of expression of the endogenous genes can also be inferred from the absence of gene expression (e.g., the absence of an mRNA or polypeptide product encoded by the gene) following the knockout, gene editing, introduction of a mutation, or inhibition of expression of the endogenous genes.

In certain aspects, a modified immune cell of the present disclosure is modified to overexpress the immune cell receptor CD226 (also referred to as PTA1 or DNAM-1). Like TIGIT, CD226 is expressed on the surface of immune cells and binds to CD155 and CD112 to mediate cell-cell adhesion. However, unlike TIGIT, CD226 lacks an ITIM domain, and is not presently believed to have immunosuppressive activity. Without wishing to be bound by theory, overexpressed CD226 may competitively inhibit binding of TIGIT to its ligands and, thereby, inhibit TIGIT-associated immune cell suppression. Overexpression of CD226 in a host immune cell can be accomplished, for example, by introducing into the immune cell an expression construct comprising a polynucleotide that encodes CD226 (or a function portion or fragment thereof) under the control of an expression control sequence (e.g., a promoter sequence), which may be an inducible or constitutively active expression control sequence. The wild-type nucleotide sequence of human CD226 can be found at NCBI Gene ID No: 10666 (updated on Oct. 14, 2018), and the full-length amino acid sequence of human CD226 can be found at UniProt ID No.: Q15762. Overexpression of CD226 can be, for example, 10%, 20%, 30%, 40%, 50%, 60%, 70%, 80%, 90%, 100%, 200%, 300%, 400%, 500%, 1000%, 5000%, 10,000%, or more (or reported as a fold-increase measure of expression) expression relative to a reference expression level of CD226, which may be from the same immune cell prior to introduction of the heterologous CD226-encoding polynucleotide or from a different immune cell of a same type that does not receive or include the heterologous CD226-encoding polynucleotide. Overexpression can be determined by, for example, qPCR, FACS, or the like.

### Methods of Making Modified Immune Cells

In further aspects, the present disclosure provides methods of making modified immune cells. In certain embodiments, methods comprise introducing, into a modified immune cell comprising a heterologous polynucleotide encoding an antigen-specific receptor that specifically binds to a tumor-associated antigen. In some embodiments, the provided methods and cells involve the further modification of the cells or further introduction so as to inhibit or prevent or reduce the activity, function, or expression of TIGIT or CD112R. In some aspects, such inhibition or prevention or reduction is effected by introducing an inhibitory nucleic acid molecule encoding a TIGIT- or CD112R-specific inhibitor, wherein the expression product of the inhibitory nucleic acid molecule inhibits endogenous TIGIT or CD112R expression in the modified cell. In further embodiments, an inhibitory nucleic acid encodes a TIGIT- or CD112R-specific antisense oligonucleotide, dsRNA molecule, siRNA molecule, esiRNA, shRNA, or any combination thereof. In certain embodiments, an inhibitory nucleic acid molecule is introduced in a modified immune cell ex vivo. In certain embodiments, the modified immune cell is further modified to overexpress CD226.

In some aspects, the provided methods and compositions are for making modified immune cells, wherein, for example, the methods may comprise introducing,—into an immune cell that comprises an inhibitory nucleic acid molecule encoding a TIGIT- or CD112R-specific inhibitor that, when expressed, inhibits endogenous TIGIT or CD112R expression in the modified cell—a heterologous polynucleotide encoding an antigen-specific receptor protein. In some embodiments, an inhibitory nucleic acid encodes CD226 and, when introduced into and expressed by the immune cell, causes or facilitates the immune cell to overexpress CD226. In some embodiments, a method comprises introducing, into an immune cell that expresses or comprises a heterologous polynucleotide encoding an antigen-specific receptor protein, a polynucleotide that encodes CD226. In some embodiments, a method comprises introducing, into an immune cell that overexpresses CD226 (or comprises a heterologous polynucleotide that encodes CD226), a heterologous polynucleotide that encodes an antigen-specific receptor protein.

In other embodiments, methods for making modified immune cells comprise introducing, into an immune cell comprising a heterologous polynucleotide encoding an antigen-specific receptor that specifically binds to a tumor-associated antigen, a chromosomal TIGIT or CD112R gene knockout or mutation (and optionally a chromosomal knockout or mutation of one or more of PD-1, CTLA4, TIM-3, LAG3, an HLA component, and a TCR component) by chromosomal editing of the immune cell, thereby generating the modified immune cell. In certain embodiments, chromosomal editing is by an endonuclease selected from a CRISPR/Cas nuclease system, a ZFN, a TALEN, or a meganuclease. In certain embodiments, a chromosomal TIGIT or CD112R gene knockout or mutation is introduced into the modified immune cell ex vivo. In certain embodiments, a modified immune cell is modified to overexpress CD226.

In still other embodiments, methods are provided that comprise introducing, into an immune cell comprising a chromosomal TIGIT or CD112R gene knockout or mutation, a heterologous polynucleotide encoding an antigen-specific receptor protein (e.g., that binds to a tumor-associated antigen of the present disclosure).

### Inhibitors of TIGIT or CD112R Expression or Activity

In some aspects, the present disclosure provides inhibitors of TIGIT or CD112R expression or activity or function and uses thereof, such as in connection with engineered immune cells such as CAR-T or TCR-engineered T cells. Such inhibitors may be useful for inhibiting TIGIT or CD112R expression or activity in a modified immune cell of the present disclosure, e.g., for use in adoptive cell therapy to treat a solid tumor. In certain embodiments, an inhibitor of TIGIT or CD112R activity, function or expression comprises an antibody or an antigen-binding fragment thereof. Terms understood by those in the art of antibody technology are each given the meaning acquired in the art, unless expressly defined differently herein.

In certain embodiments, an antibody or antigen-binding fragment thereof reduces (i.e., partially or completely) or prevents immunosuppressive signaling by TIGIT or CD112R. Reduction or prevention of TIGIT or CD112R signaling in some aspects can be achieved by directly inhibiting TIGIT or CD112R from binding to one or both of CD155 and CD112 (e.g., by competitively inhibiting a ligand-binding site of TIGIT), or by allosteric inhibition of the binding site.

In certain embodiments, an antibody comprises CDRs from, or comprises a VH and/or a VL from, or VH and/or VL having a least about 80%, 85%, 90%, 95%, 96%, 97%, 98%, or 99% identity to that of, or is selected from, any one or more of the anti-TIGIT antibodies shown in Table 1.

TABLE 1
Exemplary Anti-TIGIT Antibodies and Disclosure(s) and Sources Thereof

| Anti-TIGIT Antibody(ies) | Exemplary Disclosure/Source |
|---|---|
| RG6058 (MTIG7192A) | Garber, Nature Biotechnology |
| | 35: 487-488 (2017); Roche/Genentech |
| OMP-313M32 (etigilimab) | Garber, Nature Biotechnology |
| | 35: 487-488 (2017) |
| AB-154 | Arcus Biosciences |
| COM-902 | Compugen |
| Monoclonal Antibody to Antagonize | |
| TIGIT for Oncology (315293) | |
| Monoclonal Antibody to Antagonize | |
| TIGIT for Oncology (328189) | |
| Monoclonal Antibody to Antagonize | |
| TIGIT for Oncology (350426) | |
| ENUM-009 (326504) | |
| Monoclonal Antibody to Antagonize | |
| TIGIT for Oncology (331672) | |
| MK-7684 | Merck Sharp & Dohme Corp. |
| MBSA43 (eBioscience) | PCT Publication No. |
| | WO 2016/028656A1 |
| Abs 58, 69, 75, 133, 177, 122, 86, 180, 83, | PCT Publication No. |
| 26, 20, 147, 12, 66, 176, 96, 123, 109, | WO 2018/102746A1 |
| 149, 34, 61, 64, 105, 108, 178, 166, 29, | (see, e.g., FIGS. |
| 135, 171, 194, 184, 164, 183, 158, 55, | 2A-2C and 3A-3C) |
| 136, 39, 159, 151, 139, 107, 36, 193, 115, | |
| 106, 138, 127, 165, 155, 19, 6, 187, 179, | |
| 65, 114, 102, 94, 163, 110, 80, 92, 117, | |
| 162, 121, 195, 84, 161, 198, 116, 174, | |
| 196, 51, 91, 185, 23, 7, 95, 100, 140, 145, | |
| 150, 168, 54, 77, 43, 160, 82, 189, 17, | |
| 103, 18, 130, 132, 134, 144, 2, 47, 49, 31, | |
| 53, 40, 5, 9, 48, 4, 10, 37, 33, 42, 45, 44, | |
| 97, 81, 18B, 186, 62, 57, 192, 73, 60, 28, | |
| 32, 78, 14, 152, 72, 12B, 169, 87, 74, 172, | |
| 153, 120, 13, 113, 16, 56, 129, 50, 90, 99, | |
| 3, 148, 124, 22, 41, 119, 157, 27, 15, 191, | |
| 190, 79, 181, 146, 167, 88, 199, 71, 85, | |
| 59, 141, 68, 143, 46, 197, 175, 156, 63, | |
| 11, 182, 89, 8, 101, 25, 154, 21, 111, 118, | |
| 173, 38, 76, 131, 1, 67, 70, 170, 30, 93, | |
| 142, 104, 112, 35, 126, 125 | |
| 14A6 and antibodies having the parental | PCT Publication No. |
| consensus, clone, or humanized CDR, VH, | WO 2016/028656A1 |
| and Vk sequences thereof as set forth in | (see, e.g., Table 4) |
| SEQ ID NOs: 1-30 of WO | |
| 2016/028656A1), 28H5 and the variant | |
| sequences thereof, 31C6 and antibodies | |
| having the parental consensus or variant | |
| sequences thereof, antibodies having | |
| CDRs, a VH, and/or a VL according to | |
| any one of SEQ ID NOs: 37-52 of WO | |
| 2016/028656A1, 14H6 and the variant | |
| sequences thereof, 18G10, 11A11 | |
| Clones 2, 2C, 3, 5, 13, 13A, 13B, 13C, | PCT Publication No. |
| 13D 14, 16, 16C, 16D, 16E, 18, 21, 22, | WO 2018/160704A1 |
| 25, 25A, 25B, 25C, 25D, 25E, 27, 54 | (see, e.g, Table 3) |
| CPA.9.086, CPA.9.083, CHA.9.547.13 | PCT Publication No. |
| | WO 2018/033798A1 |
| MABs 1, 3, 4, 5, 6, 7, 8, 9, 10, 11, 12, 13, | PCT Publication No. |
| 14, 15, 16, 17, 17, 18, 19, 20, 21 | WO 2017/059095A1 |
| 313R11, 313R12, 313R14, 313R19 | PCT Publication No. |
| | WO 2016/191643A2 |
| 14B2, 13E6, 6F9, 11G11, 10C9, 16F6, | PCT Publication No. |
| 11C9, 27A9, 10D7, 20G6, 24E8, 24G1, | WO 2016/106302A1 |
| 27F1, 15A6, 4E4, 13D1, 9B11, 10B8, | |
| 22G2, 19H2, 8C8, 17G4, 25E7, 26D8, | |
| 16A8 | |

In certain embodiments, an antibody or antigen-binding fragment thereof comprises CDRs from, or comprises a VH and/or a VL from, or a VH and/or VL having a least about 80%, 85%, 90%, 95%, 96%, 97%, 98%, or 99% identity to that of, or is selected from, any one or more of the anti-CD112R antibodies shown in Table 2.

TABLE 2
Exemplary Anti-CD112R Antibodies and
Disclosure(s) and Sources Thereof

| | Exemplary |
|---|---|
| Anti-CD112R Antibody(ies) | Disclosure/Source |
| COM-701 | Compugen |
| CHA.7.518.1.H4(S241P), | PCT Publication No. |
| CHA.7.538.1.2.H4(S241P) | WO 2018/033798A1 |
| CPA.7.001-CPA.7.0050 and all antibodies | U.S. patent Pub. No. |
| therebetween | U.S. 2016/0244521A1; |
| | PCT Publication No. |
| | WO 2016/134335A1 |
| | (see, e.g., Table 4) |
| CPA.7.021, CPA.7.001, CPA.7.003, | PCT Publication No. |
| CPA.7.004, CPA.7.006, CPA.7.008, | WO 2016/134333A9 |
| CPA.7.009, CPA.7.010, CPA.7.011, | |
| CPA.7.012, CPA.7.013, CPA.7.014, | |
| CPA.7.015, CPA.7.017, CPA.7.018, | |
| CPA.7.019, CPA.7.022, CPA.7.023, | |
| CPA.7.024, CPA.7.033, CPA.7.034, | |
| CPA.7.036, CPA.7.040, CPA.7.046, | |
| CPA.7.047, CPA.7.049, CPA.7.050, | |
| CPA.7.021, CPA.7.001, CPA.7.003, | |
| CPA.7.004, CPA.7.006, CPA.7.008, | |
| CPA.7.009, CPA.7.010, CPA.7.011, | |
| CPA.7.012, CPA.7.013, CPA.7.014, | |
| CPA.7.015, CPA.7.017, CPA.7.018, | |
| CPA.7.019, CPA.7.022, CPA.7.023, | |
| CPA.7.024, CPA.7.033, CPA.7.034, | |
| CPA.7.036, CPA.7.040, CPA.7.046, | |
| CPA.7.047, CPA.7.049, CPA.7.050, | |
| CHA.7.502, CHA.7.503, CHA.7.506, | |
| CHA.7.508, CHA.7.510, CHA.7.512, | |
| CHA.7.514, CHA.7.516, CHA.7.518, | |
| CHA.7.520.1, CHA.7.520.2, CHA.7.522, | |
| CHA.7.524, CHA.7.526, CHA.7.527, | |
| CHA.7.528, CHA.7.530, CHA.7.534, | |
| CHA.7.535, CHA.7.537, CHA.7.538.1, | |
| CHA.7.538.2, CHA.7.543, CHA.7.544, | |
| CHA.7.545, CHA.7.546, CHA.7.547, | |
| CHA.7.548, CHA.7.549, CHA.7.550. | |

Alternatively or additionally, an antibody or binding fragment thereof may bind to a TIGIT or CD112R ligand (e.g., CD155 or CD112) so as to reduce or prevent (i.e. partially or completely) binding to TIGIT or CD112R. Anti-CD112 antibodies are described in, for example, PCT Publication No. WO 2017/021526, and include antibody L-14, the variable domain and CDR sequences of which are incorporated herein. Anti-CD155 antibodies include, for example: clone SKII.4 (BioLegend); and D171 (see U.S. Pat. No. 6,518,033); these antibodies and antigen-binding fragments thereof are incorporated herein by reference. In some embodiments, an antibody or antigen-binding fragment comprises CDRs from, or comprises a VH and/or a VL from, or a VH and/or VL having a least about 80%, 85%, 90%, 95%, 96%, 97%, 98%, or 99% identity to that of, or is selected from, any one or more of L-14, SKII.4, or D171.

In certain embodiments, an inhibitor of TIGIT or CD112R activity, function or expression comprises an inhibitory nucleic acid. An “inhibitory nucleic acid molecule” refers to a short, single-stranded or double-stranded nucleic acid molecule that has sequence complementary to a target gene or mRNA transcript and is capable of reducing expression of the target gene or mRNA transcript, or refers to a polynucleotide encoding such a molecule. An inhibitory nucleic acid molecule includes antisense oligonucleotides, double stranded RNA (dsRNA) molecules, small interfering RNA (siRNA molecules, shRNA molecules, and endoribonuclease-prepared siRNA (esiRNA) molecules. Accordingly, in certain embodiments, the inhibitory nucleic acid comprises an antisense oligonucleotide, dsRNA molecule, a siRNA molecule, an esiRNA, a shRNA molecule, or any combination thereof. Reduced expression may be accomplished via a variety of processes, including blocking of transcription or translation (e.g., steric hindrance), degradation of the target mRNA transcript, blocking of pre-mRNA splicing sites, blocking mRNA processing (e.g., capping, polyadenylation). In certain embodiments, inhibitory nucleic acid molecules may be used for gene knockdown methods. The genomic and mRNA sequences of TIGIT and CD112R are publicly available at, for example, the National Center for Biotechnology Information's GenBank database (Gene ID Nos. 201633 and 79037, respectively). Methods for making inhibitory nucleic acid molecules targeting mRNAs are known in the art and described, for example, in Ozcan et al. Adv. Drug Deliv. Rev. 87:108-119 (2016). Methods of inhibiting expression of a gene in an immune cell using an inhibitory nucleic acid molecule are known in the art and described, for example, in U.S. Patent Publication Nos. US 2012/0321667 and US 2007/0036773; Condomines et al., PLoS ONE 10:e0130518, 2015; Ohno et al., J. Immunother. Cancer 1:21, 2013).

In some embodiments, an inhibitor of TIGIT or CD112R comprises a heterologous polynucleotide (e.g., an expression construct) that encodes CD226 and is contained within or introduced into the modified immune cell, whereby CD226 is overexpressed by the modified immune cell and can inhibit activity, function, or expression of TIGIT or CD112R. In some embodiments, a modified immune cell is engineered (e.g., by gene-editing techniques such as those disclosed herein) to overexpress CD226.

### Kits

Also provided herein are kits that comprise one or more reagents for making or using modified immune cells and combination therapies of the present disclosure. In certain embodiments, a kit comprises: (i) a modified immune cell that expresses an antigen-specific receptor protein that specifically binds to a tumor-associated antigen; and (ii) an inhibitor of TIGIT or CD112R activity, function or expression.

In other embodiments, a kit comprises reagents for producing a modified immune cell, such as: (i) a transgenic construct encoding an antigen-specific receptor protein, which construct may be comprised in a vector capable of delivering the construct to a desired host immune cell; and (ii) a reagent for generating a chromosomal gene knockout or mutation of TIGIT or CD112R (e.g., a CRISPR-Cas system with one or more appropriate gRNA; or both (i) and (ii).

In some embodiments, a kit comprises a polynucleotide (e.g., an expression construct) that encodes CD226, such that when the polynucleotide is introduced into and expressed by a host immune cell (e.g., expressing or comprising a heterologous polynucleotide encoding an antigen-specific receptor), CD226 is overexpressed by the host immune cell. In some embodiments, the kit further comprises the modified immune cell into which the CD226-encoding polynucleotide is to be introduced.

In any of the herein disclosed embodiments, a kit can further comprise instructions for producing the modified immune cell and/or for administering one or both of the modified immune cell and the inhibitor of TIGIT or CD112R activity, function or expression to treat a disease (e.g., a solid tumor-associated disease, such as a solid cancer).

### Methods of Treatment

The compositions disclosed herein may be useful in therapies, such as immunotherapies, for treating a solid tumor. In further aspects, the present disclosure provides immunotherapy methods for treating a solid tumor, wherein the methods comprise administering to a subject having a solid tumor an effective amount of (a) a modified immune cell comprising a heterologous polynucleotide encoding an antigen-specific receptor that specifically binds to a tumor-associated antigen and (b) an inhibitor of TIGIT or (i.e., and/or) CD112R activity, function or expression.

In other aspects, the present disclosure provides immunotherapy methods for treating a solid tumor, wherein the methods comprise administering to a subject having a solid tumor an effective amount of an inhibitor of T cell Ig and ITIM domain (TIGIT) or CD112R activity, function or expression, wherein the subject has previously received a modified immune cell comprising a heterologous polynucleotide encoding an antigen-specific receptor that specifically binds to a tumor-associated antigen.

In still other aspects, the present disclosure provides immunotherapy methods for treating a solid tumor, wherein the methods comprising administering to a subject having a solid tumor an effective amount of a modified immune cell comprising a heterologous polynucleotide encoding an antigen-specific receptor that specifically binds to a tumor-associated antigen wherein the subject has previously received an inhibitor of T cell Ig and ITIM domain (TIGIT) or CD112R activity, function or expression.

In certain embodiments, an antigen-specific receptor comprises a CAR or a TCR.

In certain embodiments, an inhibitor of TIGIT or CD112R activity, function or expression comprises an antibody or an antigen-binding fragment thereof. In certain embodiments, an inhibitor of TIGIT or CD112R activity, function or expression comprises an inhibitory nucleic acid.

In certain embodiments, a modified immune cell is modified to overexpress CD226.

In any of the embodiments disclosed herein, a modified immune cell and an inhibitor of TIGIT or CD112R expression or activity can be administered to a subject in any order (i.e., simultaneously, concurrently, or in any sequence) or in any combination.

In yet another aspect, immunotherapy methods for treating a solid tumor are provided, wherein the methods comprise administering to a subject having a solid tumor an effective amount of a modified immune cell targeting a tumor-associated antigen, wherein the modified immune cell comprises (a) a heterologous polynucleotide encoding an antigen-specific receptor that specifically binds to the tumor-associated antigen and (b) a chromosomal TIGIT or CD112R gene knockout. In certain embodiments, a chromosomal TIGIT or CD112R gene knockout is made by chromosomal editing of the immune cell.

In any of the embodiments disclosed herein, a modified immune cell can comprise a human immune cell. In some embodiments, a modified immune cell can comprise a T cell (e.g., CD4⁺, CD8⁺, stem cell memory, or the like), a Treg cell, a NK cell, or an NK-T cell. In certain embodiments, an immune cell is a CD4⁺ T cell or a CD8⁺ T cell. In certain embodiments, a modified immune cell is modified to overexpress CD226. In certain embodiments, a modified immune system cell is allogeneic, autologous, or syngeneic to the subject. In any of the embodiments described herein, a modified immune cell can be generated in vitro or ex vivo.

The level of an immune response against a solid tumor (e.g., a CTL (cytotoxic T lymphocyte) immune response) may be determined by any one of numerous immunological methods described herein. The level of a CTL immune response may be determined prior to and following administration of any one of the herein described antigen-specific binding receptors expressed by, for example, a T cell. Cytotoxicity assays for determining CTL activity may be performed using any one of several techniques and methods (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

Exemplary cancers that can form tumors and can be targeted with the methods of this disclosure include sarcomas and carcinomas, including, for example, chondrosarcoma; fibrosarcoma (fibroblastic sarcoma); Dermatofibrosarcoma protuberans (DF SP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; synovial sarcoma; Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); a lung carcinoma (e.g., Adenocarcinoma, Squamous Cell Carcinoma (Epidermoid Carcinoma), Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma).

In certain embodiments, methods of the present disclosure target a solid tumor formed by a cancer selected from an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In certain embodiments, methods of the present disclosure are useful for treating triple-negative breast cancer, mantle cell lymphoma, acute lymphocytic leukemia, non-small-cell lung cancer, or any combination thereof.

In certain embodiments, a solid tumor treatable by the presently disclosed methods expresses a tumor-associated antigen selected from ROR1, EGFR, EGFRvIII, EGP-2, EGP-40, GD2, GD3, HPV E6, HPV E7, HER2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, MAGE-A3, MAGE-A4, PSA, PRAME, ephrin A2, ephrin B2, an NKG2D, NY-ESO-1, TAG-72, mesothelin, NY-ESO, SSX-2, SSX-3, 5T4, BCMA, FAP, Carbonic anhydrase 9, ERBB2, BRAF^{V600E}, CEA, or any combination thereof. In certain embodiments, a solid tumor treatable by the presently disclosed methods expresses or overexpresses a ROR1 antigen.

Antigen-specific T cell responses can be determined by comparison of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a tumor antigen (e.g., peptide, polypeptide, glycan, or the like). A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-administration) data.

An effective amount, e.g., a therapeutically n effective amount of cells in a composition is at least one cell (for example, one binding protein modified CD8⁺ T cell subpopulation; one binding protein modified CD4⁺ T cell subpopulation) or is more typically greater than 10² cells, for example, up to 10⁶, up to 10⁷, up to 10⁸ cells, up to 10⁹ cells, or more than 10¹⁰ cells. In certain embodiments, the cells are administered in a range from about 10⁴ to about 10¹⁰ cells/m², preferably in a range of about 10⁵ to about 10⁹ cells/m².

In some embodiments, a modified immune cell is administered to a subject at a dose comprising up to about 3.3×10⁵ cells/kg. In some embodiments, a modified immune cell is administered to a subject at a dose comprising at least about 3.3×10⁵ cells/kg.

In some embodiments, a modified immune cell is administered to a subject at a dose comprising up to about 1×10⁶ cells/kg. In some embodiments, a modified immune cell is administered to a subject at a dose comprising least about 1×10⁶ cells/kg.

In some embodiments, a modified immune cell is administered to a subject at a dose comprising up to about 3.3×10⁶ cells/kg. In some embodiments, a modified immune cell is administered to a subject at a dose comprising at least about 3.3×10⁶ cells/kg.

In some embodiments, a modified immune cell is administered to a subject at a dose comprising up to about 1×10⁷ cells/kg. In some embodiments, a modified immune cell is administered to a subject at a dose comprising at least about 1×10⁷ cells/kg.

In certain embodiments, a modified immune cell is administered to a subject at a dose comprising up to about 5×10⁴ cells/kg, 5×10⁵ cells/kg, 5×10⁶ cells/kg, or up to about 5×10⁷ cells/kg. In certain embodiments, a modified immune cell is administered to a subject at a dose comprising at least about 5×10⁴ cells/kg, 5×10⁵ cells/kg, 5×10⁶ cells/kg, or up to about 5×10⁷ cells/kg.

The number of cells will depend upon the ultimate use for which the composition is intended as well the type of cells included therein. For example, cells modified to contain an antigen-specific receptor will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells.

Unit doses are also provided herein which comprise a therapeutically effective amount of the host cells (e.g., modified immune cells comprising a polynucleotide of the present disclosure). In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a subject sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% modified CD4⁺ T cells, combined with (ii) a composition comprising at least about 70% modified CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose comprises equal, or approximately equal numbers of modified CD45RA⁻ CD3⁺ CD8⁺ and modified CD45RA− CD3⁺ CD4⁺ T_{M} cells.

Modified immune cells as described herein may be administered to a subject in a pharmaceutically or physiologically acceptable or suitable excipient or carrier. Pharmaceutically acceptable excipients are biologically compatible vehicles, e.g., physiological saline, which are described in greater detail herein, that are suitable for administration to a human or other non-human mammalian subject. A therapeutically effective dose, in the context of adoptive cell therapy, is an amount of host cells (expressing a binding protein according to the present disclosure) used in adoptive transfer that is capable of producing a clinically desirable result (e.g., a cytotoxic T cell response) in a statistically significant manner) in a treated human or non-human mammal. As is well known in the medical arts, the dosage for any one patient depends upon many factors, including the patient's size, weight, body surface area, age, the particular therapy to be administered, sex, time and route of administration, general health, and other drugs being administered concurrently.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's disease, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

The pharmaceutical compositions described herein may be presented in unit-dose or multi-dose containers, such as sealed ampoules or vials. Such containers may be frozen to preserve the stability of the formulation until. The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired therapeutic effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which may be performed using samples obtained from a subject before and after treatment.

In certain embodiments, immunotherapy methods of the present disclosure comprise combination therapies comprising administering to a subject having a solid tumor a modified immune cell (and an inhibitor of TIGIT or CD112R expression or activity, where appropriate) and one or more additional agents. For example, in certain embodiments, a method of this disclosure further comprises administering an agent that targets another immune cell molecule or functionality to increase or improve antitumor activity in the subject. In certain embodiments, a method of this disclosure further comprises administering to the subject an effective amount of DEL-106. DEL-106 is a fusion protein comprising an IL-2 mutein fused to an antibody Fc domain, and is used to preferentially upregulate Treg cells.

In certain embodiments, a modified immune cell (and an inhibitor of TIGIT or CD112R expression or activity, where appropriate) is used with an inhibitor of an immune suppression component or an agonist of a stimulatory immune checkpoint molecule, as described herein, to enhance an antitumor response by the immune system and to, ultimately, treat a tumor or associated cancer.

As used herein, the term “immune suppression component” or “immunosuppression component” refers to one or more cells, proteins, molecules, compounds or complexes providing inhibitory signals to assist in controlling or suppressing an immune response. For example, immune suppression components include those molecules that partially or totally block immune stimulation; decrease, prevent or delay immune activation; or increase, activate, or up regulate immune suppression. Exemplary immunosuppression component targets are described in further detail herein and include PD-1, PD-L1, PD-L2, LAG3, CTLA4, B7-H3, B7-H4, CD244/2B4, HVEM, BTLA, CD160, TIM3, GALS, KIR, PVR1G (CD112R), PVRL2, adenosine, A2aR, immunosuppressive cytokines (e.g., IL-10, IL-4, IL-1RA, IL-35), IDO, arginase, VISTA, LAIR1, CEACAM-1, CEACAM-3, CEACAM-5, Treg cells, or any combination thereof.

An inhibitor of an immune suppression component may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a modified immune cell (and an inhibitor of TIGIT or CD112R expression or activity, where appropriate) with one or more inhibitor of any one of the following immune suppression components, singly or in any combination.

In certain embodiments, a modified immune cell (and an inhibitor of TIGIT or CD112R expression or activity, where appropriate) is used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558 or any combination thereof. In further embodiments, a modified immune cell (and an inhibitor of TIGIT or CD112R expression or activity, where appropriate) is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In further embodiments, a modified immune cell (and an inhibitor of TIGIT or CD112R expression or activity, where appropriate) is used in combination with a LAG3 inhibitor, such as LAG525, IMP321, IMP701, 9H12, BMS-986016, or any combination thereof.

In certain embodiments, a modified immune cell (and an inhibitor of TIGIT or CD112R expression or activity, where appropriate) is used in combination with an inhibitor of CTLA4. In particular embodiments, a modified immune cell (and an inhibitor of TIGIT or CD112R expression or activity, where appropriate) is used in combination with a CTLA4 specific antibody or binding fragment thereof, such as ipilimumab, tremelimumab, CTLA4-Ig fusion proteins (e.g., abatacept, belatacept), or any combination thereof.

In further embodiments, a modified immune cell (and an inhibitor of TIGIT or CD112R expression or activity, where appropriate) is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO 2016/40724 and WO 2013/025779.

In some embodiments, a modified immune cell (and an inhibitor of TIGIT or CD112R expression or activity, where appropriate) is used in combination with an inhibitor of CD244.

In certain embodiments, a modified immune cell (and an inhibitor of TIGIT or CD112R expression or activity, where appropriate) is used in combination with an inhibitor of BLTA, HVEM, CD160, or any combination thereof. Anti CD-160 antibodies are described in, for example, PCT Publication No. WO 2010/084158.

In more embodiments, a modified immune cell (and an inhibitor of TIGIT or CD112R expression or activity, where appropriate) is used in combination with an inhibitor of TIM3.

In still more embodiments, a modified immune cell (and an inhibitor of TIGIT or CD112R expression or activity, where appropriate) is used in combination with an inhibitor of Gal9.

In certain embodiments, a modified immune cell (and an inhibitor of TIGIT or CD112R expression or activity, where appropriate) is used in combination with an inhibitor of adenosine signaling, such as a decoy adenosine receptor.

In further embodiments, a modified immune cell (and an inhibitor of TIGIT or CD112R expression or activity, where appropriate) is used in combination with an inhibitor of A2aR.

In still further embodiments, a modified immune cell (and an inhibitor of TIGIT or CD112R expression or activity, where appropriate) is used in combination with an inhibitor of KIR, such as lirilumab (BMS-986015).

In yet further embodiments, a modified immune cell (and an inhibitor of TIGIT or CD112R expression or activity, where appropriate) is used in combination with an inhibitor of an inhibitory cytokine (typically, a cytokine other than TGFβ) or Treg development or activity.

In some embodiments, a modified immune cell (and an inhibitor of TIGIT or CD112R expression or activity, where appropriate) is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a modified immune cell (and an inhibitor of TIGIT or CD112R expression or activity, where appropriate) is used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof.

In further embodiments, a modified immune cell (and an inhibitor of TIGIT or CD112R expression or activity, where appropriate) is used in combination with an inhibitor of VISTA, such as CA-170 (Curis, Lexington, Mass.).

In further embodiments, a modified immune cell (and an inhibitor of TIGIT or CD112R expression or activity, where appropriate) is used in combination with a LAIR1 inhibitor.

In more embodiments, a modified immune cell (and an inhibitor of TIGIT or CD112R expression or activity, where appropriate) is used in combination with an inhibitor of CEACAM-1, CEACAM-3, CEACAM-5, or any combination thereof.

In certain embodiments, a modified immune cell (and an inhibitor of TIGIT or CD112R expression or activity, where appropriate) is used in combination with an agent that increases the activity (i.e., is an agonist) of a stimulatory immune checkpoint molecule. For example, a modified immune cell (and an inhibitor of TIGIT or CD112R expression or activity, where appropriate) can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2), an agonist of GITR (such as, for example, humanized monoclonal antibodies described in PCT Patent Publication No. WO 2016/054638), or an agonist of ICOS (CD278) (such as, for example, GSK3359609, mAb 88.2, JTX-2011, Icos 145-1, or Icos 314-8), or any combination thereof. In any of the embodiments disclosed herein, a method may comprise administering a modified immune cell (and an inhibitor of TIGIT or CD112R expression or activity, where appropriate) with one or more agonist of a stimulatory immune checkpoint molecule, including any of the foregoing, singly or in any combination.

In other embodiments, a method of this disclosure further comprises administering a secondary therapy comprising one or more of: an antibody or antigen-binding fragment specific for a cancer antigen expressed by the solid tumor being targeted; a chemotherapeutic agent; surgery; radiation therapy treatment; a cytokine; an RNA interference therapy, or any combination thereof.

Exemplary monoclonal antibodies useful in cancer therapies include, for example, monoclonal antibodies described in Galluzzi et al., Oncotarget 5(24):12472-12508, 2014, which antibodies are incorporated by reference in their entirety.

In certain embodiments, a combination therapy method comprises administering a modified immune cell (and an inhibitor of TIGIT or CD112R activity, function or expression, where appropriate) and further administering a radiation treatment or a surgery. Radiation therapy is includes X-ray therapies, such as gamma-irradiation, and radiopharmaceutical therapies. Surgeries and surgical techniques appropriate to treating a given cancer or non-inflamed solid tumor may be used in a subject in combination with a modified immune cell of this disclosure.

In certain embodiments, a combination therapy method comprises administering a modified immune cell (and an inhibitor of TIGIT or CD112R expression or activity, where appropriate) and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines can be used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination.

Another cancer therapy approach involves reducing expression of oncogenes and other genes needed for growth, maintenance, proliferation, and immune evasion by cancer cells. RNA interference, and in particular the use of microRNAs (miRNAs) small inhibitory RNAs (siRNAs) provides an approach for knocking down expression of cancer genes. See, e.g., Larsson et al., Cancer Treat. Rev. 16(55):128-135, 2017.

In any of the embodiments disclosed herein, any of the therapeutic agents (e.g., a modified immune cell, an inhibitor of TIGIT or CD112R expression or activity, an inhibitor of an immune suppression component, an agonist of a stimulatory immune checkpoint molecule, an antitumor lymphocyte, a chemotherapeutic agent, a radiation therapy, a surgery, a cytokine, or an inhibitory RNA) may be administered once or more than once to the subject over the course of a treatment, and, in combinations, may be administered to the subject in any order (e.g., simultaneously, concurrently, or in any sequence) or any combination. An appropriate dose, suitable duration, and frequency of administration of the compositions will be determined by such factors as a condition of the patient; size, type, spread, growth, and severity of the tumor or cancer; particular form of the active ingredient; and the method of administration.

In certain embodiments, a plurality of doses of a modified immune cell as described herein is administered to the subject, which may be administered at intervals between administrations of about two to about four weeks. In further embodiments, a cytokine (e.g., IL-2, IL-15, IL-21) is administered sequentially, provided that the subject was administered the recombinant host cell at least three or four times before cytokine administration. In certain embodiments, a cytokine is administered concurrently with the host cell. In certain embodiments, a cytokine is administered subcutaneously.

In still further embodiments, a subject being treated is further receiving immunosuppressive therapy, such as calcineurin inhibitors, corticosteroids, microtubule inhibitors, low dose of a mycophenolic acid prodrug, or any combination thereof. In yet further embodiments, a subject being treated has received a non-myeloablative or a myeloablative hematopoietic cell transplant, wherein the treatment may be administered at least two to at least three months after the non-myeloablative hematopoietic cell transplant.

An effective amount of a therapeutic or pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

### EXAMPLES

### Example 1

### TIGIT Expression in ROR-1 Car T Cells Following Adoptive Transfer

A Phase I single-center study was conducted using immunotherapy to treat patients with advanced receptor tyrosine kinase-like orphan receptor 1 positive (ROR1⁺) chronic lymphocytic leukemia (CLL), mantle cell lymphoma (MCL), acute lymphoblastic leukemia (ALL), stage IV non-small cell lung cancer (NSCLC), or triple negative breast cancer (TNBC).

ROR1-specific CARs were constructed using VL and VH chain segments of the 2A2, R12, and R11 mAbs (specific for ROR1). For example, variable domain sequences for R11 and R12 can be found in Yang et al. (Plos One 6:e21018, 2011), and variable domain sequences for 2A2 can be found in U.S. Pat. No. 9,316,646. Each pair of variable domains from 2A2, R12, and R11 were individually linked by a (G₄S)₃ (SEQ ID NO:48) variable region linker peptide. The ROR1 scFv was linked to a spacer derived from IgG4-Fc (Uniprot Database: P01861, SEQ ID NO:49) comprising a “hinge-CH3” fragment (119 amino acids from SEQ ID NO:49) or “hinge” only (12 amino acids, SEQ. ID NO:46 or 47) spacer. The spacer of SEQ ID NO:46 contains a S→P substitution within the “hinge” domain located at position 108 of the native IgG4-Fc protein. The scFv-spacer was linked to a 27 amino acid transmembrane domain of human CD28 (Uniprot: P10747) and to a signaling module comprising either (i) a 41-amino acid cytoplasmic domain from the human CD28 (optionally with an LL→GG substitution located at positions 186-187 of the native CD28 protein) or (ii) the 42 amino-acid cytoplasmic domain of human 4-1BB (Uniprot: Q07011), each of which was linked to a 112-amino acid cytoplasmic domain of isoform 3 of human CD3ζ (Uniprot: P20963). The construct includes, downstream of the encoded CAR, a Thosea asigna virus (T2A) self-cleaving element encoded by ctcgagggcg gcggagaggg cagaggaagt cttctaacat gcggtgacgt ggaggagaatcccggcccta gg (SEQ ID NO:66), and a truncated EGFR (tEGFR; the nucleic acid sequence encoding tEGFR can be found in PCT Application No. PCT/US2014/072007, which tEGFR-encoding sequence is incorporated herein by reference) to serve as a transduction, selection and in vivo tracking marker for CAR-modified immune cells. Codon-optimized nucleotide sequences encoding each transgene were synthesized (Life Technologies) and cloned into the epHIV7 lentiviral vector.

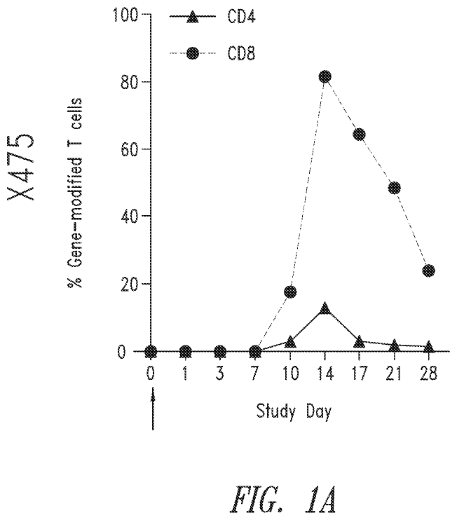

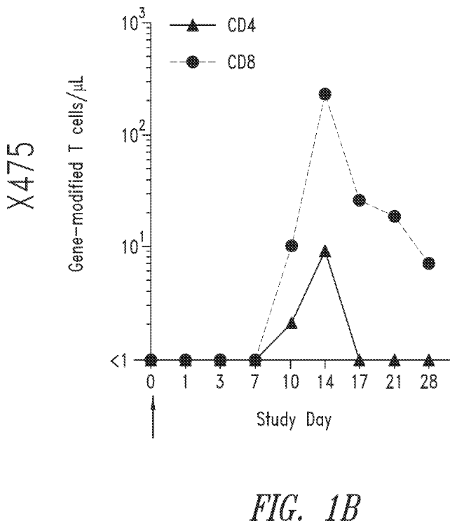

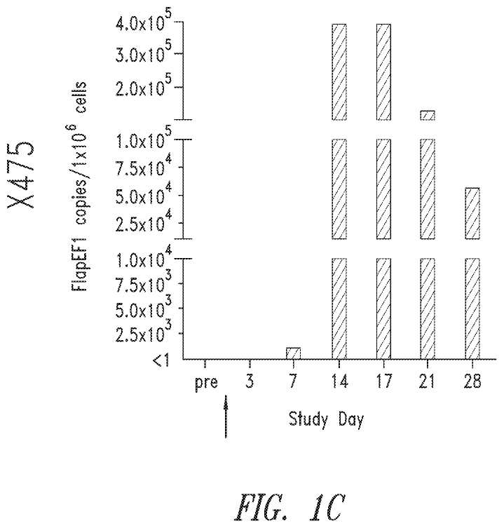

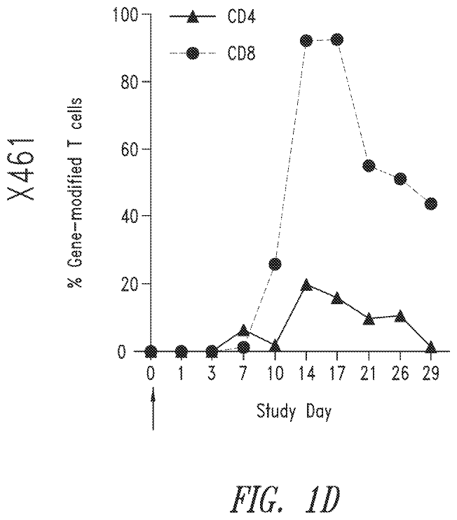

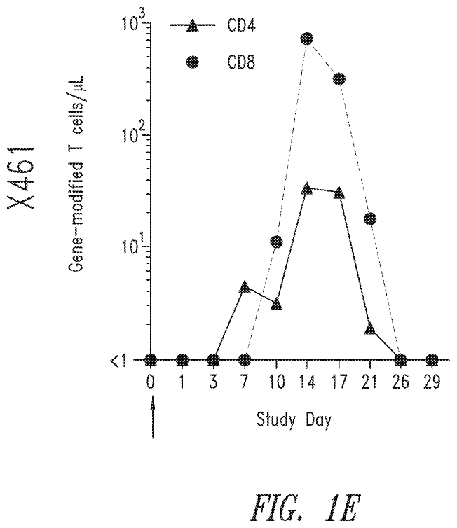

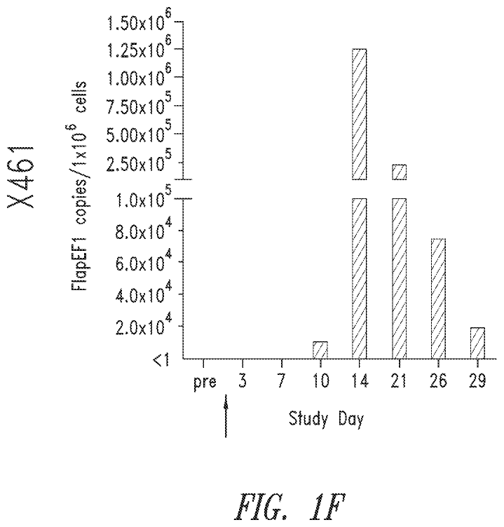

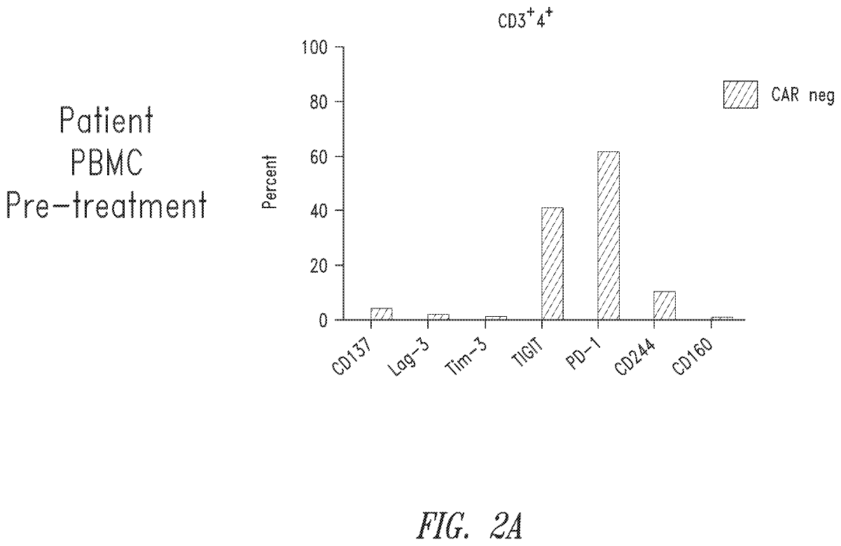

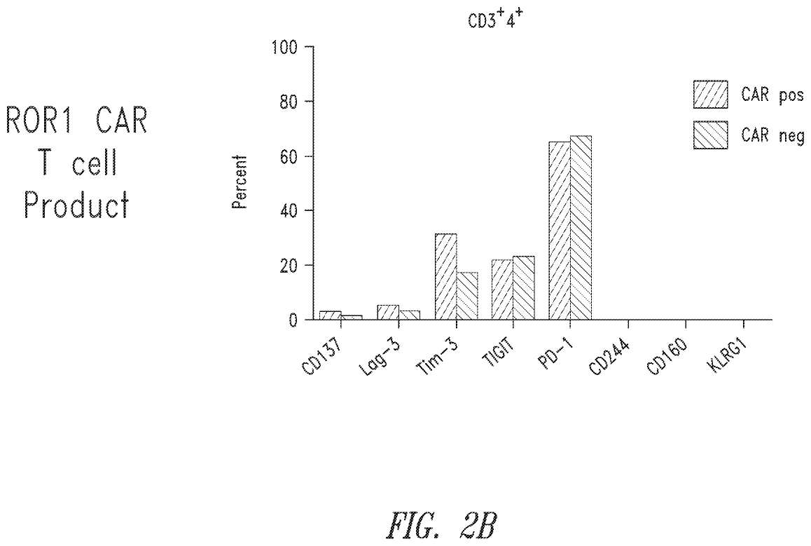

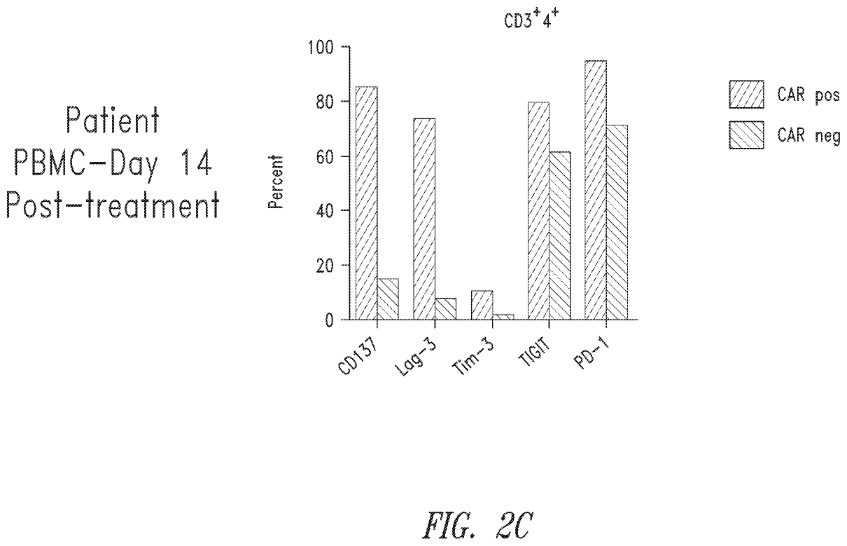

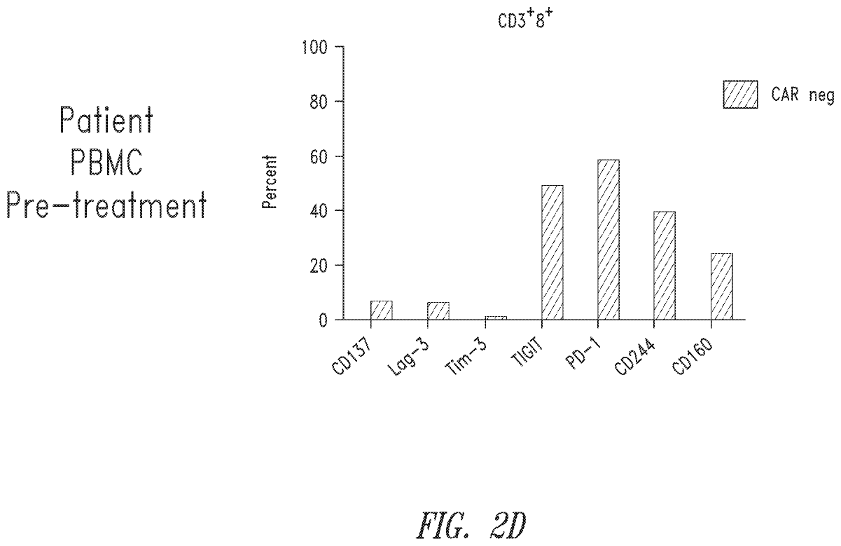

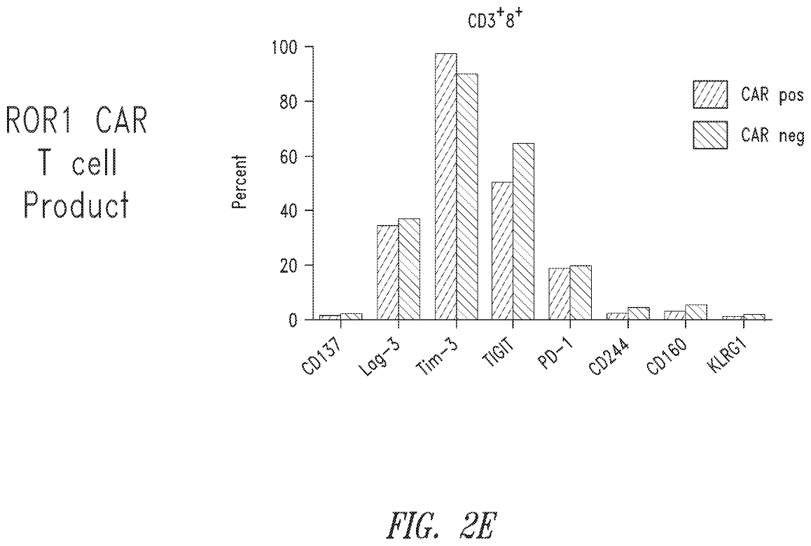

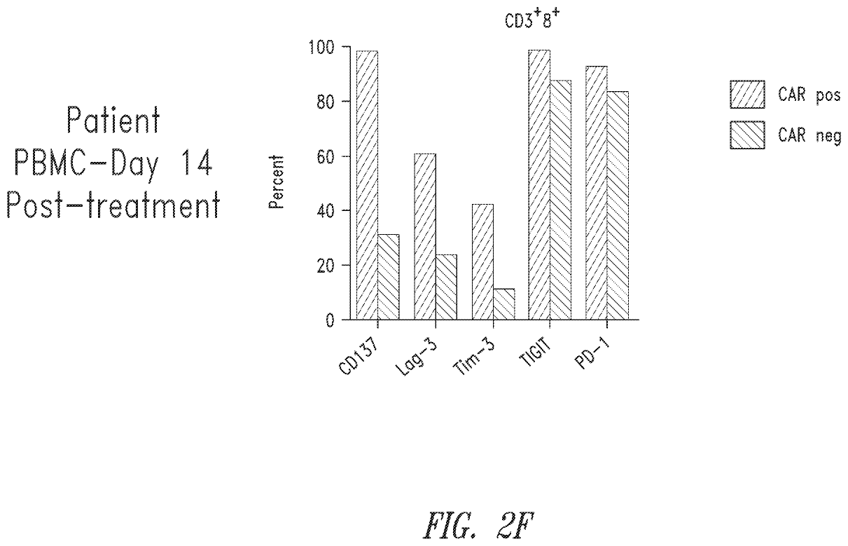

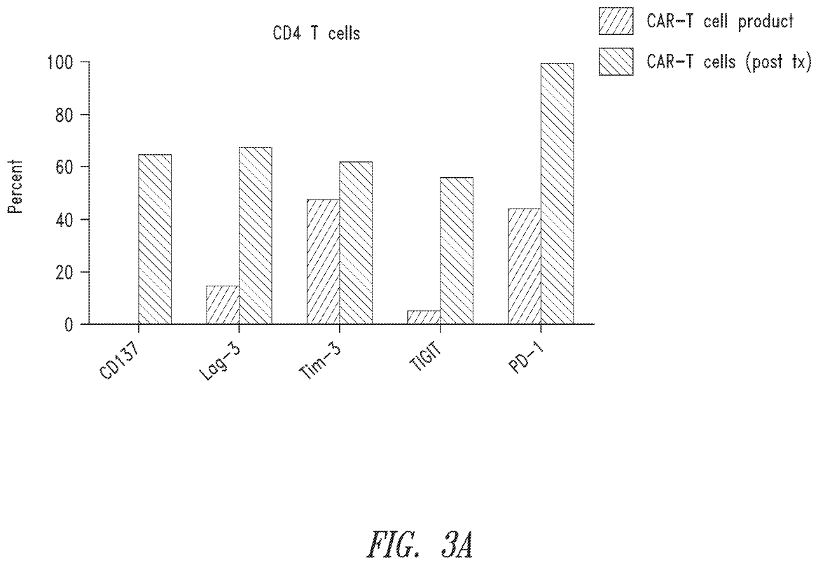

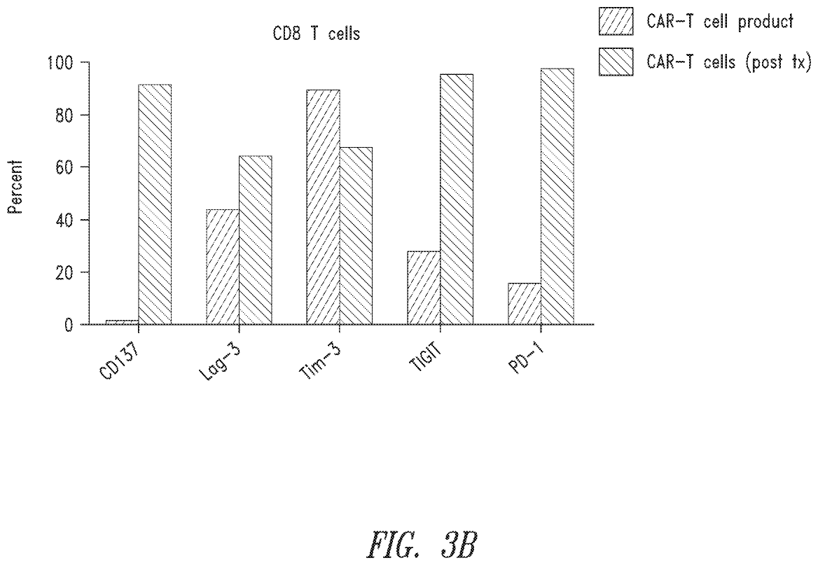

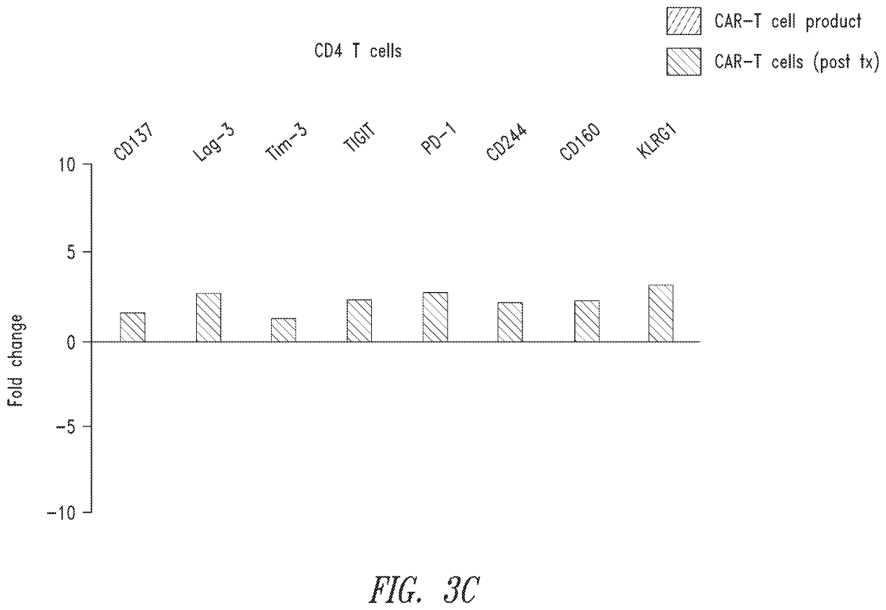

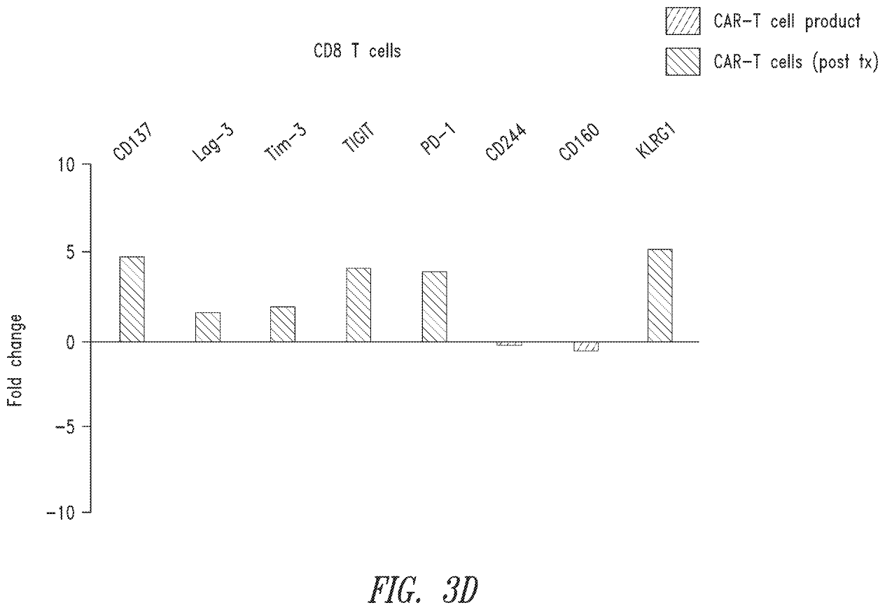

Autologous T cells were isolated from patient PBMCs and transduced ex vivo with a construct encoding an anti-ROR1 chimeric antigen receptor (CAR) and examined for functionality (proliferation, cytokine release) in response to stimulation with a ROR1-expressing tumor cell. Thereafter, CAR T cells were expanded in vitro and administered to the patients via intravenous infusion. The CAR T cells expanded in the patients after adoptive transfer and could be measured by flow cytometry and q-PCR for vector sequences (FIGS. 1A-1F). Samples were taken from treated patients at the peak of in vivo expansion to measure phenotype, assess function, and determine antitumor persistence. Flow cytometry and gene expression profiling was performed to characterize gene expression in the cells. TIGIT was expressed at low levels in pre-treatment patient PBMC (FIGS. 2A and 2D) and only in a small fraction of the administered CAR⁺ T product (FIGS. 2B and 2E), but showed increased expression by flow cytometry at the peak of in vivo expansion (day +14) after adoptive transfer (FIGS. 2C and 2F). Similar data by flow cytometry was obtained from a second patient, and was confirmed by gene expression analysis showing upregulation of TIGIT on the persisting CAR⁺ T cells at the peak of in vivo expansion after adoptive transfer (FIGS. 3A-3D). Upregulation of CD112R mRNA was also observed on CAR T cells present in blood after infusion compared to the infusion product [CD8⁺ CD112R-1.87-fold increase (peak CART cells versus infusion product); CD4⁺ CD112R-1.95-fold increase (peak CAR T cells versus infusion product)] (data not shown).

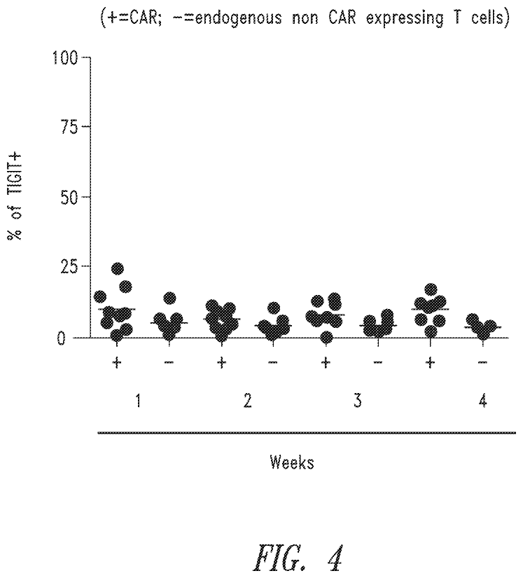

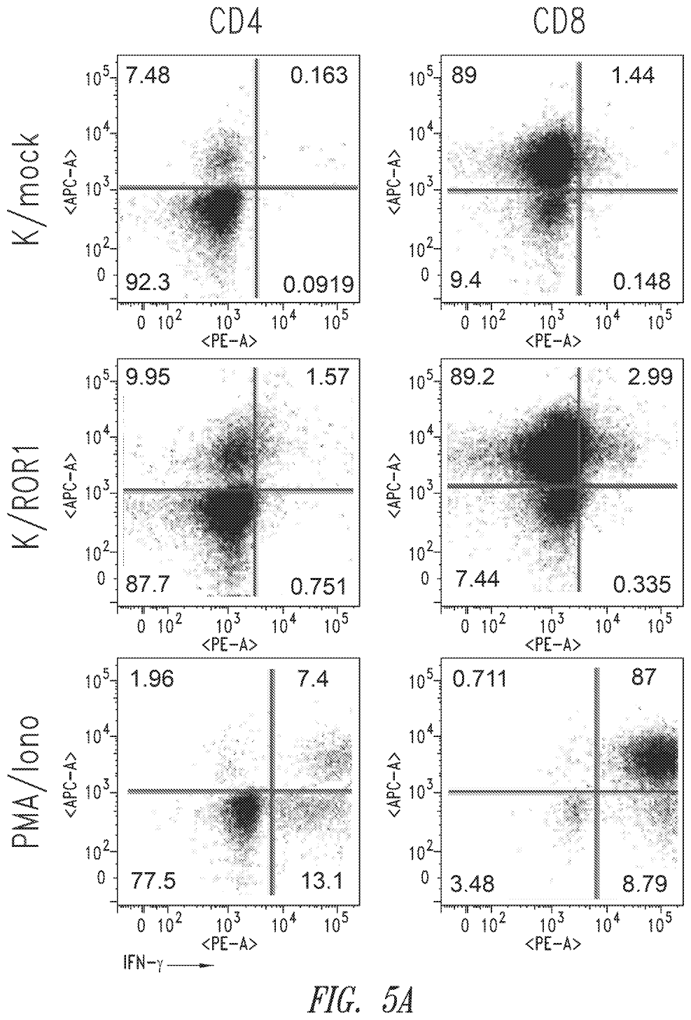

Surprisingly, upregulation of TIGIT to the same degree or at the same frequency of cells was not observed at any time point in vivo in CART cells targeting CD19 that were transferred to patients with CD19⁺ hematological malignancies (FIG. 4). Notably, CD19-specific CAR⁺ T cells, which have impressive antitumor efficacy against CD19⁺ malignancies, do not upregulate TIGIT.

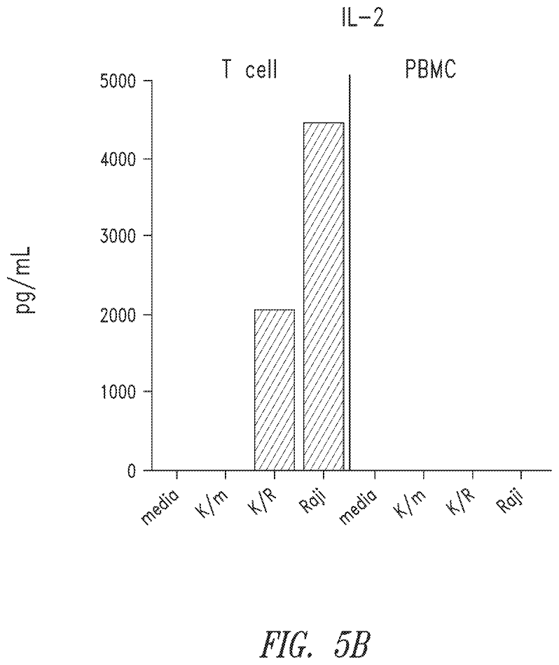

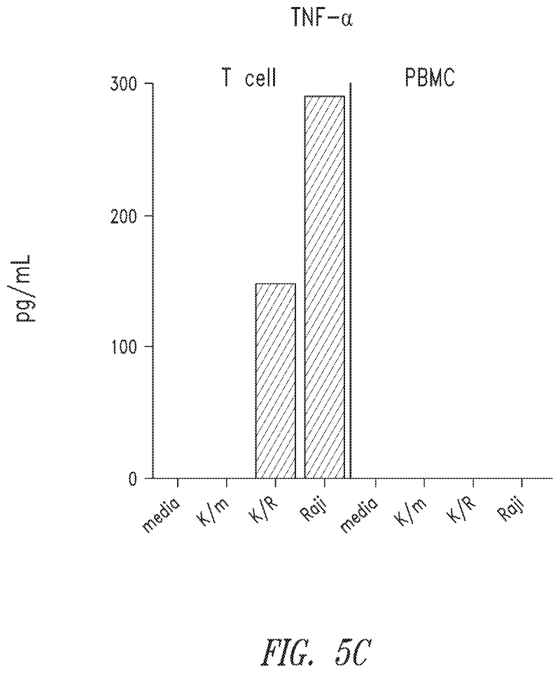

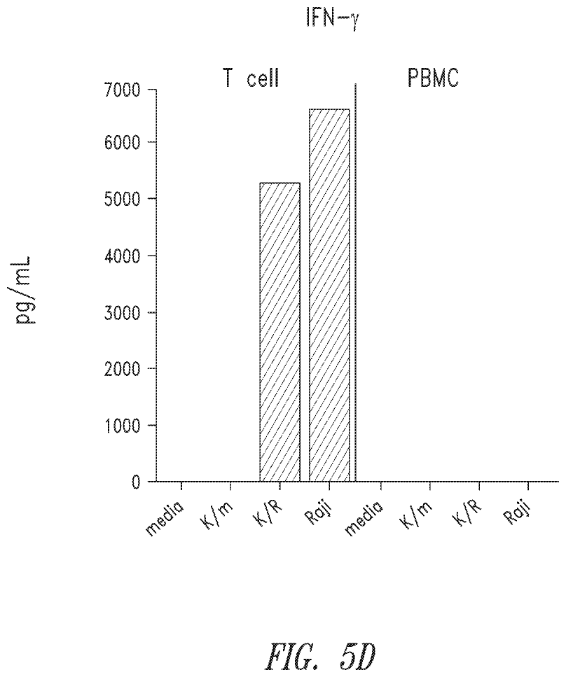

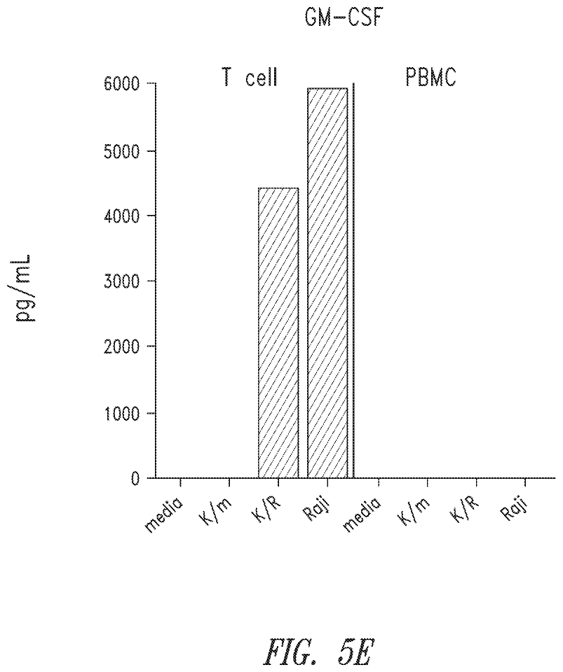

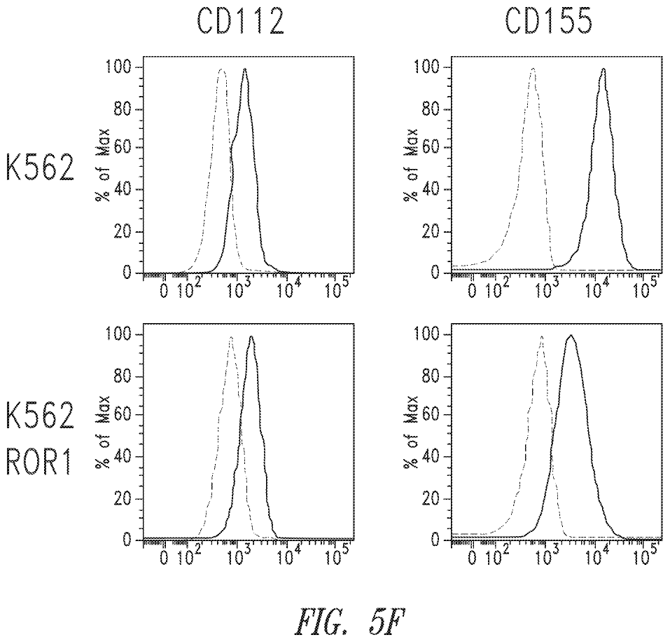

The functionality of (1) ROR1-specific CART cells at the time of administration to patients with lung or breast cancer, and (2) ROR1-specific CAR T cells that persisted in the patient, was assessed by measuring the ability of the cells to produce cytokines and to proliferate after re-stimulation with ROR1-expressing tumor cells. The CAR T cells that persisted in vivo and expressed high levels of TIGIT exhibited a reduced ability to produce cytokines in response to re-stimulation with cells expressing the ROR1 antigen compared with the ROR CAR T cell product (i.e., not exposed to the tumor), as shown by intracellular cytokine staining for interferon gamma (FIG. 5A), and by measuring the levels of cytokines in the supernatant (FIGS. 5B-5E). The antigen-expressing K562 cells and K562 transfectants used in this in vitro assay express the TIGIT and CD112R ligands CD155 and CD112 (FIG. 5F). These data demonstrate that upregulation of TIGIT and CD112R correlates with a loss of T cell function, and that inhibition of TIGIT/CD112R upregulation and/or binding to its ligands can be used as a supplemental treatment of solid tumors with CAR T cells to minimize immune suppression and sustain the function of tumor antigen-specific CAR T cells in vivo.

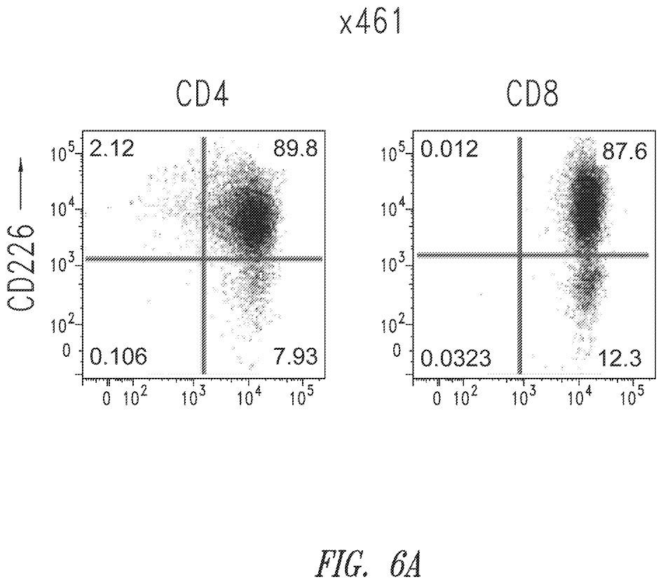

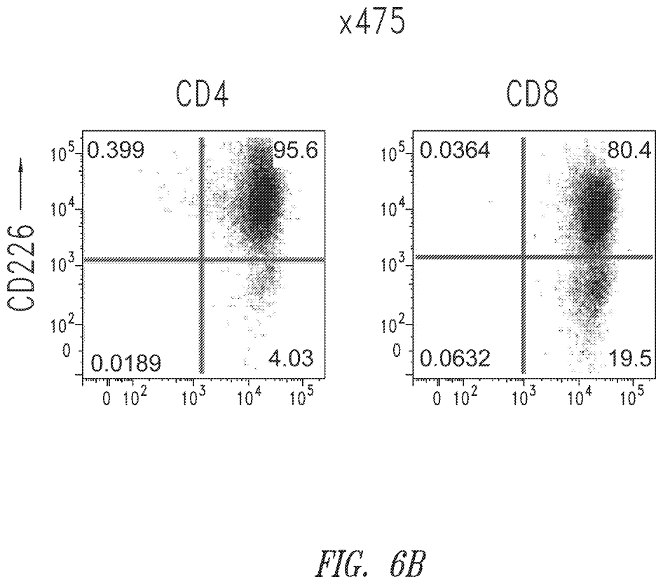

TIGIT is believed to function as an inhibitory receptor, in part, by competing with CD226 for binding to CD155. The expression of CD226 was examined on ROR1 CAR T cells prepared from patients X461 and X475 by flow cytometry; both CD4⁺ and CD8⁺ T cells from each patient were shown to express high levels of CD226 (FIGS. 6A and 6B).

To examine the effects of other immunosuppressive molecules, CAR T cell expression of TIGIT, CD112R, and other co-inhibitory molecules is monitored and analyzed over the course of the trial. Transcriptional profiles and functionality of the CAR T cells are further examined prior to infusion and at various points after infusion.

### Example 2

### In Vitro Model of TIGIT Expression in ROR1 Car T Cells

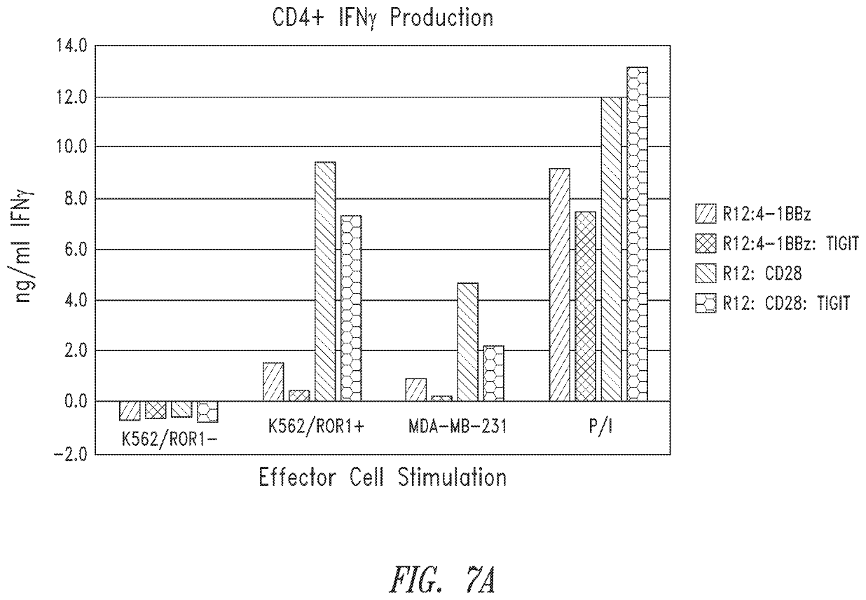

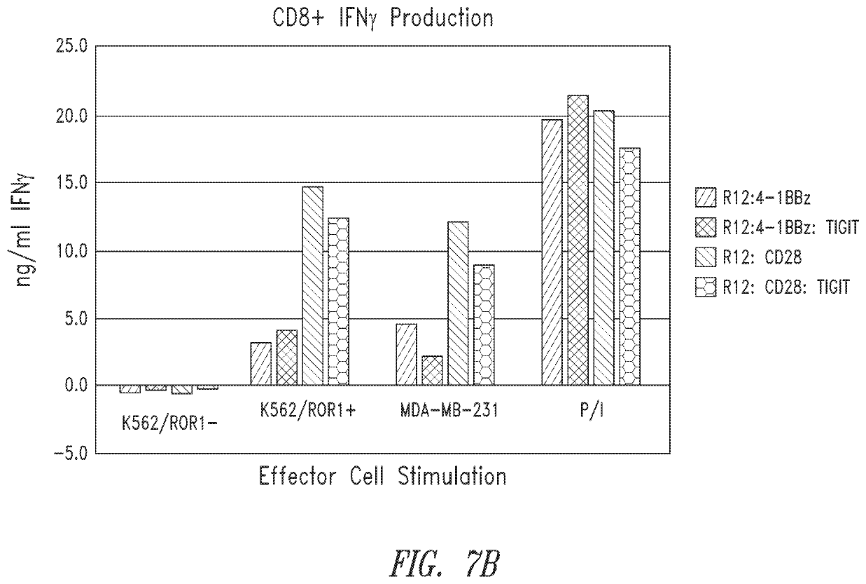

Model tumor antigen-specific CD4⁺ or CD8⁺ CAR T cells were developed to constitutively overexpress TIGIT by incorporating TIGIT downstream of a 2A element in the ROR1 CAR cassette. These CAR T cells were then exposed to tumor cells that express TIGIT ligands (CD155 and CD112) and functionality was examined. In contrast to control CAR T cells (i.e., lacking constitutive expression of TIGIT), the model cells demonstrated cell-intrinsic functional suppression when exposed to the tumor cells in vitro, as demonstrated by a reduction in IFN-γ production (see FIGS. 7A and 7B).

### Example 3

### In Vitro Study of TIGIT Signaling Activity in ROR-1 Car T Cells

To further investigate the effect of TIGIT and CD112R on immunotherapy, ROR1-specific CAR T cells are cultured in vitro with cells expressing the TIGIT/CD112R ligands CD155 and CD112. Expression profiling is performed and functionality (proliferation and cytokine release following stimulation with ROR1 antigen) is analyzed. Subsequently, blocking antibodies against TIGIT, CD112R, CD112, and/or CD155 are introduced alone or in combination with antibodies that block other known co-inhibitory receptors, including PD-1, and CAR T cell functionality is again analyzed.

### Example 4

### In Vivo Model of CAR T Cell Therapy with TIGIT Inhibition

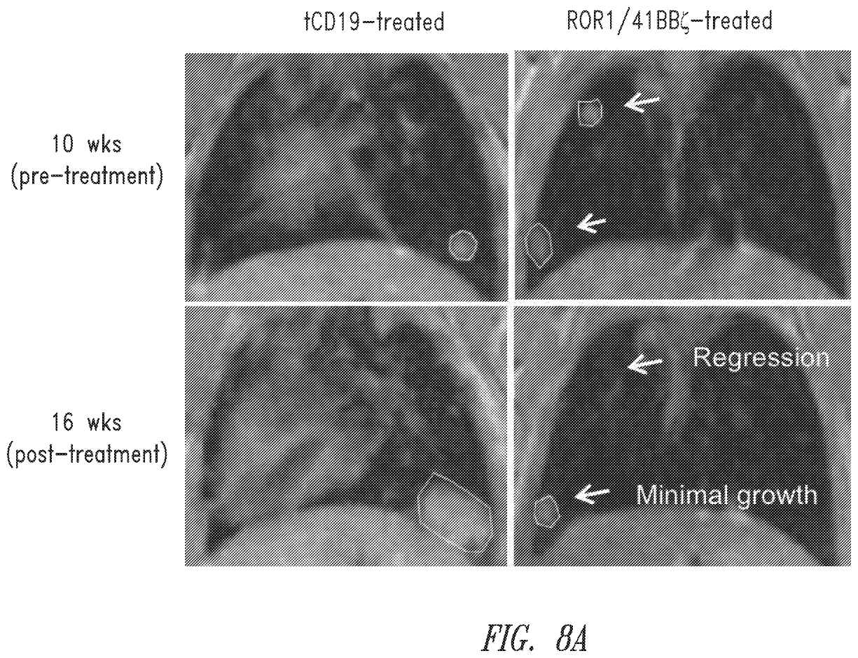

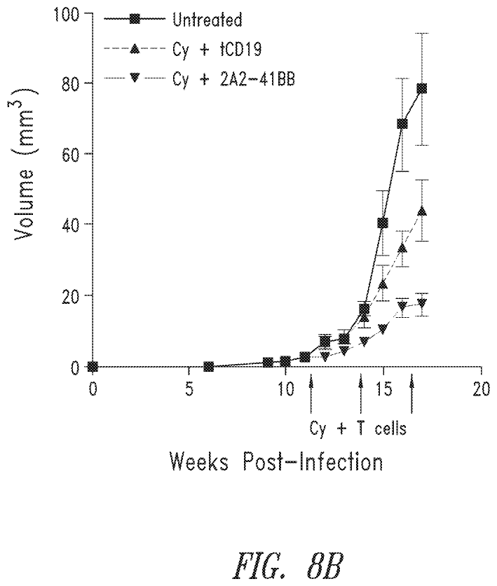

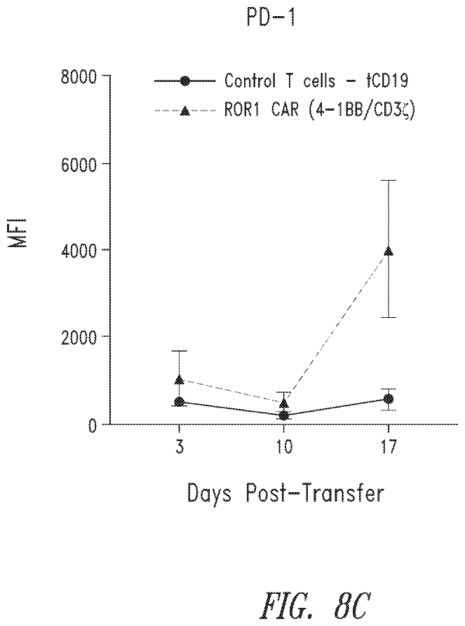

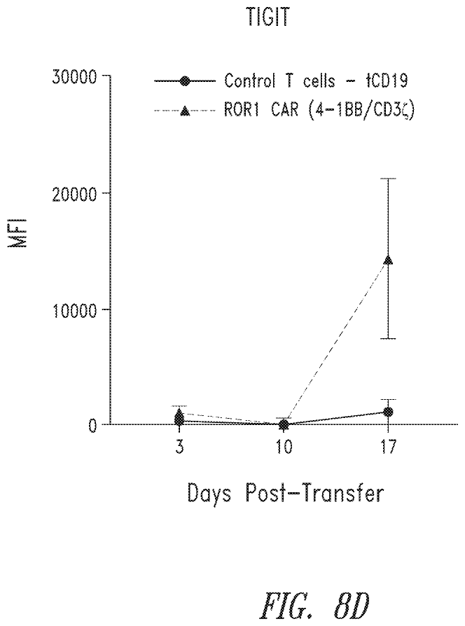

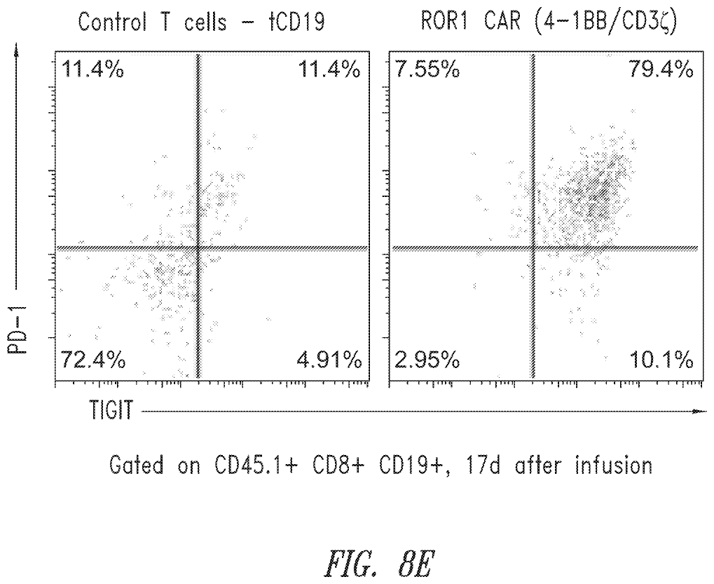

To examine the effect of anti-ROR1 CAR T cell therapy, an in vivo xenograft tumor model that is ROR1⁺ was developed. Briefly, the “KP” mouse model (LSL-Kras^{+/-} p53^{fl/fl}) of lung adenocarcinoma (Jackson et al., Cancer Res. 65(22):10280 (2005)) was transfected intratracheally with a lentivirus carrying Cre recombinase and human ROR1 (Dupage et al., Nat. Protoc. 4(7):1064 (2009)), to produce a KP ROR1⁺ mouse model that replicates a ROR1⁺ human tumor in the mice. KP ROR1⁺ mice were monitored for tumor nodule growth (using Magnetic Resonance Imaging), and CAR T cells expressing a ROR1 (R12)-specific construct as described in Example 1 were administered once the animals developed tumor nodules of ≥1 mm³. Anti-ROR1 CAR T cells alone exhibited significant but transient control of tumor growth in the mice (FIGS. 8A and 8B). The administered anti-ROR1 CAR⁺ T cells showed an increased level of PD-1 and, surprisingly, TIGIT expression after administration to the animals, similar to that observed in patients (FIGS. 8C-8E).

To further study the effects of TIGIT expression on CAR T cell therapy, KP ROR1⁺ mice having tumor nodules ≥1 mm³ are administered ROR1-specific CAR T cells (a) alone or (b) in combination with an anti-TIGIT antibody (3-6 mice per study group). ROR1-specific CAR T cells are removed and examined for functionality at various time points following administration to the ROR1⁺ KP mice. Also, throughout treatment, the amount of bound TIGIT on the ROR1-specific CAR T cells is assayed, and tumor size and serum cytokine levels are monitored to determine efficacy. Optionally, overall tumor size and morphology are also examined to detect changes (e.g., reduction in tumor size, integrity, or both) following the ROR1-specific CAR T therapy. Survival is also determined over time.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Patent Application No. 62/575,326, filed on Oct. 20, 2017, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An immunotherapy method for treating a solid tumor, the method comprising administering to a subject having a solid tumor an effective amount of (a) a modified immune cell comprising a heterologous polynucleotide encoding an antigen-specific receptor that specifically binds to a tumor-associated antigen and (b) an inhibitor of T cell Ig and ITIM domain (TIGIT) and/or CD112R activity, function or expression.

2. An immunotherapy method for treating a solid tumor, the method comprising administering to a subject having a solid tumor an effective amount of an inhibitor of T cell Ig and ITIM domain (TIGIT) and/or CD112R activity, function or expression, wherein the subject has previously received a modified immune cell comprising a heterologous polynucleotide encoding an antigen-specific receptor that specifically binds to a tumor-associated antigen.

3. An immunotherapy method for treating a solid tumor, the method comprising administering to a subject having a solid tumor an effective amount of a modified immune cell comprising a heterologous polynucleotide encoding an antigen-specific receptor that specifically binds to a tumor-associated antigen wherein the subject has previously received an inhibitor of T cell Ig and ITIM domain (TIGIT) and/or CD112R activity, function or expression.

4. The immunotherapy method of claim 1, wherein the inhibitor of TIGIT and/or CD112R activity, function or expression comprises (a) an antibody or an antigen-binding fragment thereof, and/or (b) an inhibitory nucleic acid.

5.-10. (canceled)

11. The immunotherapy method of claim 1, wherein the inhibitor of TIGIT and/or CD112R activity, function, or expression comprises a heterologous polynucleotide that encodes CD226, whereby the modified immune cell comprising the heterologous CD226-encoding polynucleotide overexpress CD226.

12. The immunotherapy method of claim 1, wherein the modified immune cell comprises a T cell, a Treg cell, a NK cell, a NK-T cell, or any combination thereof.

13. The immunotherapy method of claim 1, wherein the modified immune cell is a CD4⁺ T cell and/or a CD8⁺ T cell.

14. (canceled)

15. The immunotherapy method of claim 1, wherein the antigen-specific receptor comprises a chimeric antigen receptor (CAR), a T cell receptor (TCR), or both.

16.-21. (canceled)

22. The immunotherapy method of claim 1, wherein the solid tumor is a carcinoma or a sarcoma.

23. The immunotherapy method of claim 1, wherein the solid tumor is selected from chondrosarcoma; fibrosarcoma (fibroblastic sarcoma); Dermatofibrosarcoma protuberans (DF SP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

24. The immunotherapy method of claim 1, wherein the solid tumor is selected from Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); a lung carcinoma (e.g., Adenocarcinoma, Squamous Cell Carcinoma (Epidermoid Carcinoma), Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma).

25. The immunotherapy method of claim 1, wherein the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

26. The immunotherapy method of claim 1, wherein the solid tumor is a triple-negative breast cancer, a non-small-cell lung cancer, a mantle cell lymphoma, or an acute lymphocytic leukemia.

27. (canceled)

28. The immunotherapy method of claim 1, further comprising administering to the subject an immunosuppression component inhibitor targeting PD-1; PD-L1; PD-L2; CTLA4; B7-H3; H7-H4; VISTA; BTLA; KIR; LAG3; GALS; TIM-3; A2AR; an immunosuppressive cytokine; CD244 (2B4); CD160; PVRIG (CD112R); arginase; indoleamine 2,3 dioxygenase (DO); IL-10; IL-4; IL-1RA; IL-35; LAIR1; CEACAM-1; CEACAM-3; CEACAM-5; Treg cells; or any combination thereof.

29. (canceled)

30. The immunotherapy method of claim 1, further comprising administering to the subject an agonist of a stimulatory immune checkpoint molecule.

31. (canceled)

32. The immunotherapy method of claim 1, wherein the tumor-associated antigen comprises Receptor tyrosine kinase-like orphan receptor 1 (ROR1), EGFR, EGFRvIII, EGP-2, EGP-40, GD2, GD3, HPV E6, HPV E7, HER2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, MAGE-A3, MAGE-A4, PRAME, PSA, ephrin A2, ephrin B2, an NKG2D, NY-ESO-1, TAG-72, mesothelin, NY-ESO, SSX-2, SSX-3, 5T4, BCMA, FAP, Carbonic anhydrase 9, ERBB2, BRAFV600E, CEA, or any combination thereof.

33.-35. (canceled)

36. A genetically modified immune cell, comprising (a) a heterologous polynucleotide encoding an antigen-specific receptor that specifically binds to a tumor-associated antigen, and (b) a chromosomal TIGIT and/or CD112R gene knockout or mutation.

37. A genetically modified immune cell, comprising (a) a heterologous polynucleotide encoding an antigen-specific receptor that specifically binds to a tumor-associated antigen, and (b) a heterologous polynucleotide that encodes CD226, whereby the modified immune cell overexpress CD226.

38.-49. (canceled)

50. A pharmaceutical composition comprising the genetically modified immune cell of claim 36 and a pharmaceutically acceptable carrier, excipient, or diluent.

51. A kit, comprising:
a modified immune cell that expresses an antigen-specific receptor protein that specifically binds to a tumor-associated antigen; and
(ii) an inhibitor of TIGIT and/or CD112R activity, function or expression.

52. A kit, comprising:
(i) one or more reagent for producing a modified immune cell that comprises a heterologous polynucleotide encoding an antigen-specific binding protein that specifically binds to a tumor-associated antigen; and
(ii) one or more reagent for generating a chromosomal gene knockout or mutation of TIGIT and/or CD112R in the immune cell.

53. A kit, comprising:
(i) a modified immune cell that expresses an antigen-specific receptor protein that specifically binds to a tumor-associated antigen; and
(ii) a polynucleotide that encodes CD226, such that when the polynucleotide is capable of being introduced into and expressed by the modified immune cell, whereupon CD226 is overexpressed by the modified immune cell.

54.-64. (canceled)

65. An immunotherapy method for treating a solid tumor, the method comprising administering to a subject having a solid tumor an effective amount of the genetically modified immune cell of claim 36.
